SEQUENCE LISTING (6 sequences)

SEQ ID NO: 1 - protein, 5 aa, unidentified
YIGSR

SEQ ID NO: 2 - protein, 5 aa, synthetic construct
IKVAV

SEQ ID NO: 3 - protein, 6 aa, synthetic construct
SIKVAV

SEQ ID NO: 4 - protein, 5 aa, synthetic construct
  SITE 5: note = C-terminus linked via an ethylene diamine bridge to the C-terminus of X3 of the structure X1-X2-X3; wherein X1 is N-acetyl-N-myristoyl-3,4-diaminobutanoic acid, X2 is 3-amino-butanoic acid, X3 is a modified aspartic acid which is modified in that 3-amino-gamma-butyrolactone is linked to the carbonyl group of the aspartic acid side chain
YIGSR

SEQ ID NO: 5 - protein, 5 aa, unidentified
  SITE 5: note = C-terminus linked via an ethylene diamine bridge to the C-terminus of X3 of the structure X1-X2-X3; wherein X1 is N-acetyl-N-myristoyl-3,4-diaminobutanoic acid, X2 is 3-amino-butanoic acid, X3 is a modified aspartic acid which is modified in that the 3-amino-group of 3,7-diamino-heptanoic acid is linked to the carbonyl group of the aspartic acid side chain
  SITE 1: note = acetylation of the N terminus
YIGSR

SEQ ID NO: 6 - protein, 5 aa, unidentified
  SITE 5: note = C-terminus linked via an ethylene diamine bridge to the C-terminus of X2 of the structure X1-X2; wherein X1 is N-acetyl-N-myristoyl-3,4-diaminobutanoic acid, X2 is a modified aspartic acid which is modified in that the 3-amino-group of 3,7-diamino-heptanoic acid is linked to the carbonyl group of the aspartic acid side chain
  SITE 1: note = acetylation of the N terminus
YIGSR

(19) United States
(12) Patent Application Publication
(10) Pub. No.: US 2026/0108612 A1
(43) Pub. Date: Apr. 23, 2026

(54) BETA-PEPTIDES AND BETA-PEPTIDE HYDROGELS

(71) Applicant: MONASH UNIVERSITY, Clayton, Victoria (AU)

(72) Inventors: Marie-Isabel Aguilar, Clayton, Victoria (AU); Mark Del Borgo, Clayton, Victoria (AU); Ketav Kulkarni, Clayton, Victoria (AU); Andrew Hong, Clayton, Victoria (AU); John Forsythe, Clayton, Victoria (AU); Bradley Broughton, Clayton, Victoria (AU); Helena C. Parkington, Clayton, Victoria (AU); Seyedeh Mahnaz Modarresi Saryazdi, Clayton, Victoria (AU); Meg McFetridge, Clayton, Victoria (AU)

(21) Appl. No.: 18/865,896

(22) PCT Filed: May 17, 2023
(86) PCT No.: PCT/AU2023/050418

§ 371 Date: Nov. 14, 2024

(30) Foreign Application Priority Data
May 17, 2022 (AU) 2022901321

(51) Int. Cl. A61K 47/42; A61K 9/00; A61K 9/06; A61K 35/28; A61P 9/10; C07K 5/09; C07K 5/107

(52) U.S. Cl. CPC A61K 47/42; A61K 9/0019; A61K 9/06; A61K 35/28; A61P 9/10; C07K 5/0817; C07K 5/1016

(57) ABSTRACT

The present invention relates to β-peptides and hydrogels comprising the β-peptides. The hydrogels may further comprise a therapeutic cargo encapsulated within the hydrogel. Methods of preparing the hydrogels, and methods for the use of the hydrogels are also described.

This patent application claims priority from Australian provisional patent application no. 2022901321 filed on 17 May 2022, the entire contents of which are incorporated herein by this reference.

### FIELD

The present invention relates to β-peptides. The present invention also relates to hydrogels comprising the β-peptides. The hydrogels may further comprise a therapeutic cargo encapsulated within the hydrogel. The present invention further relates to methods of preparing the hydrogels, and methods for the use of the hydrogels.

### BACKGROUND

There has been significant interest in the development of materials and systems for the delivery of therapeutics which avoid the issues of conventional administration.

An area of particular interest is in the delivery of cell therapies, including stem cell therapies. Stem cell therapy may be useful to promote the repair of diseased or damaged biological tissue. For example, stem cell therapy has shown potential in the treatment of stroke. Ischemic stroke is a debilitating disease caused by interruption of blood flow to the brain, resulting in neuronal cell death and injury of cerebral tissue. Preclinical studies have shown that exogenous stem cell therapy can promote tissue regeneration and improve functional outcomes.

While stem cell therapies have been shown to be promising as evidenced by recent clinical trials being conducted worldwide, there are limitations that prevent its adoption as a mainstream treatment. Typically, a large number of cells will be damaged or killed during the delivery process, making it difficult to control the number of cells administered to a patient. Poor integration and low cell viability within the host tissue can also hamper effective treatment of the patients.

Accordingly, there is a need for delivery systems that can be used in the delivery of cells such as stem cells to harness their potential therapeutic benefits.

### SUMMARY

The present invention is predicated in part on the discovery of β-peptides that spontaneously self-assemble in aqueous solution into fibres, leading to the formation of a fibrillar network that absorbs water and forms hydrogels. The hydrogels are capable of acting as delivery systems for therapeutic cargo, including cells.

In a first aspect of the invention there is provided a β-peptide of formula (I):

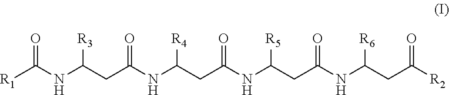

wherein
R₁ is selected from —C₁₋₂₀alkyl, —C₂₋₂₀alkenyl, —C₂₋₂₀alkynyl, —OC₁₋₂₀alkyl, —OC₂₋₂₀alkenyl and —OC₂₋₂₀alkynyl;
R₂ is selected from —H, —OR₁₀ₐ, —SR₁₀ₐ, —N(R₁₁ₐ)₂, —NH(C═NH)NH₂, cycloalkyl, aryl, heterocyclyl, and heteroaryl, where each cycloalkyl, aryl, heterocyclyl, and heteroaryl is unsubstituted or is substituted with one or more groups selected from —OH, —NH₂, —NHC₁₋₃alkyl, —OC₁₋₃alkyl, —SH, —SC₁₋₃alkyl, —CO₂H, —CO₂C₁₋₃alkyl, —CONH₂ and —CONHC₁₋₃alkyl, or R₂ has the following structure (A):

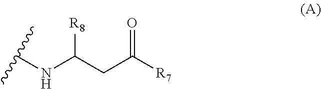

R₃, R₄, R₅, R₆ and R₈ are each independently selected from —H, —R₉, —C₁₋₆alkylR₉, —C₂₋₆alkenylR₉ and —C₂₋₆alkynylR₉;
or R₂ and R₆ together form a heterocyclic ring;
R₇ is selected from —H, —OR₁₀ₐ, —SR₁₀ₐ, —N(R₁₁ₐ)₂, —NH(C═NH)NH₂, cycloalkyl, aryl, heterocyclyl, and heteroaryl, where each cycloalkyl, aryl, heterocyclyl, and heteroaryl is unsubstituted or is substituted with one or more groups selected from —OH, —NH₂, —NHC₁₋₃alkyl, —OC₁₋₃alkyl, —SH, —SC₁₋₃alkyl, —CO₂H, —CO₂C₁₋₃alkyl, —CONH₂ and —CONHC₁₋₃alkyl;
or R₇ and R₈ together form a heterocyclic ring;
R₉ is selected from —H, —OR₁₀, —SR₁₀, —N(R₁₁)₂, —C(O)R₁₂, —NH(C═NH)NH₂, cycloalkyl, aryl, heterocyclyl, and heteroaryl, where each cycloalkyl, aryl, heterocyclyl, and heteroaryl is unsubstituted or is substituted with one or more groups selected from —OH, —NH₂, —NHC₁₋₃alkyl, —OC₁₋₃alkyl, —SH, —SC₁₋₃alkyl, —CO₂H, —CO₂C₁₋₃alkyl, —CONH₂ and —CONHC₁₋₃alkyl;
R₁₀ is selected from —H, —C₁₋₆alkyl, —C₂₋₆alkenyl, —C₂₋₆alkynyl, —C(O)C₁₋₂₀alkyl, —C(O)C₁₋₂₀alkenyl, —C(O)C₁₋₂₀alkynyl, —C(O)C₁₋₆alkylR₁₄, an α-peptide having 1 to 10 α-amino acid residues and an imaging agent;
R₁₀ₐ is selected from —H, —C₁₋₆alkyl, —C₂₋₆alkenyl, —C₂₋₆alkynyl, —C(O)C₁₋₆alkyl, —C(O)C₂₋₆alkenyl, and —C(O)C₂₋₆alkynyl;
each R₁₁ is independently selected from —H, —C₁₋₆alkyl, —C₁₋₆alkylN(R₁₃)₂, —C₁₋₆alkylNR₁₃R₁₄, —C₂₋₆alkenyl, —C₂₋₆alkynyl, —C(O)C₁₋₂₀alkyl, —C(O)C₁₋₂₀alkenyl, —C(O)C₁₋₂₀alkynyl, —C(O)C₁₋₆alkylR₁₄, an α-peptide having 1 to 10 α-amino acid residues and an imaging agent;
each R₁₁ₐ is independently selected from —H, —C₁₋₆alkyl, —C₂₋₆alkenyl, —C₂₋₆alkynyl, —C(O)C₁₋₆alkyl, —C(O)C₂₋₆alkenyl, —C(O)C₂₋₆alkynyl;
R₁₂ is selected from —OH, —OR₁₀ and —N(R₁₁)₂;
each R₁₃ is independently selected from —H and —C₁₋₆alkyl;
R₁₄ is an α-peptide having 1 to 10 α-amino acid residues or an imaging agent;
or a pharmaceutically acceptable salt thereof;
and wherein one of R₁, R₂, R₃, R₄, R₅, R₆ and R₈ comprise a C₈₋₂₀alkyl group, a C₈₋₂₀alkenyl group or a C₈₋₂₀alkynyl group.

In a second aspect of the invention there is provided a hydrogel comprising one or more β-peptides of formula (I) described herein.

In a third aspect of the invention there is provided a method of preparing a hydrogel comprising the steps of:
providing one or more β-peptides of formula (I) described herein; and
mixing the one or more β-peptides of formula (I) in an aqueous solution to form a hydrogel composition;
wherein the hydrogel composition gelates to form the hydrogel.

In a fourth aspect of the invention there is provided a method of preparing a hydrogel comprising the steps of:
providing one or more β-peptides of formula (I) described herein;
providing a therapeutic cargo; and
mixing the one or more β-peptides of formula (I) and the therapeutic cargo in an aqueous solution to form a hydrogel composition;
wherein the hydrogel composition gelates to form the hydrogel and wherein the therapeutic cargo is encapsulated within the hydrogel.

In a fifth aspect of the invention there is provided a method for treating stroke comprising administering to a patient in need thereof a hydrogel comprising:
one or more β-peptides of formula (I) described herein; and
a stem cell encapsulated within the hydrogel.

In a sixth aspect of the invention there is provided the use of a hydrogel comprising:
one or more β-peptides of formula (I) described herein; and
stem cells encapsulated within the hydrogel,
in the manufacture of a medicament for treating stroke.

In a seventh aspect of the invention there is provided the use of a hydrogel comprising:
one or more β-peptides of formula (I) described herein; and
a stem cell encapsulated within the hydrogel,
for treating stroke.

In an eighth aspect of the invention there is provided a hydrogel comprising
one or more β-peptides of formula (I) described herein; and
a cell encapsulated within the hydrogel.

### BRIEF DESCRIPTION OF THE FIGURES

FIG. 1 shows atomic force microscopy images of β-peptides Ac-Az(Myr)AKS*-Lac (1) (FIG. 1a) and Ac-Az(Myr)AX(RGD)S*-Lac (2) (FIG. 1b) dissolved in ultrapure water at 0.25 mg/mL concentration (scale bar indicates 2 m).

FIG. 2 shows a graphical representation of the percentage weight change over time of hydrogels prepared using 10 mg/mL 100% 1 (squares), 50% 1/50% 2 (triangles) and 100% 2 (circles) over time in DMEM/F12 at 37° C. (error bars represent standard deviation, n=3).

FIG. 3 shows a graphical representation of the relative cell viabilities of human amnion epithelial cells encapsulated within hydrogels prepared using 10 mg/mL 100% 1, 95% ⅕% 2, 90% 1/10% 2,75% 1/25% 2, 67% 1/33% 2, 50% 1/50% 2, 25% 1/75% 2, and 100% 2 after 3 days via MTS assay (viability relative to 100% 1, error bars represent standard deviation, ****=p<0.0001, n=4).

FIG. 4 shows a graphical representation of the relative cell viabilities of human amnion epithelial cells encapsulated within non-injected and injected 10 mg/mL 50% 1/50% 2 hydrogel-cell systems after 3 days compared to non-injected and injected DMEM/F12 controls (error bars represent standard deviation, **=p<0.005, ****=p<0.0001, n=4).

FIG. 5 shows a graphical representation of functional performance in a hanging wire test (FIG. 5a) and a cylinder test (FIG. 5b) of mice treated with vehicle, hydrogel, human amnion epithelial cells or hydrogel-encapsulated human amnion epithelial cells compared to sham in a model of photothrombotic stroke (*=p<0.05 compared to all other stroke+treatment groups, n.s.=no significant difference between each of the treatment groups (p>0.5)).

FIG. 6 shows a graphical representation of quantification of infarct volume for mice treated with vehicle, hydrogel, human amnion epithelial cells or hydrogel-encapsulated human amnion epithelial cells compared to sham (n.s.=no significant difference between each of the treatment groups (p>0.5)).

FIG. 7 shows a representative fluorescence image of a hydrogel (arrow) containing human amnion epithelial cells (white spots inside hydrogel) located within the infarct of a mouse brain following photothrombotic stroke. Glial scar area is indicated between the dotted lines. Scale bar=100 m

FIG. 8 shows a graphical representation of the average number of human amnion epithelial cells counted inside the infarct in the brains of mice treated with cells alone or treated with a hydrogel-cell system in a model of photothrombotic stroke (*=p<0.05).

FIG. 9 shows graphical representations of quantification of average glial scar width (FIG. 9a, *=p<0.05) and density of activated astrocytes within the glial scar (FIG. 9b, *=p<0.05) in the infarct of the brain of mice treated with vehicle, hydrogel, human amnion epithelial cells or hydrogel-encapsulated human amnion epithelial cells compared to sham in a model of photothrombotic stroke.

FIG. 10 shows a graphical representation of cell viabilities of human amnion epithelial cells within hydrogels prepared using 50% 1/50% 2 compared to hydrogels prepared using 50% 8/50% 9 in a Live Dead assay.

FIG. 11 shows fluorescent microscopy images of 3D encapsulation and Live Dead assay of mesenchymal stem cells encapsulated within hydrogels prepared using 80% 8/20% 9 (FIG. 1a), 80% 8/20% 10 (FIG. 11b), 50% 8/50% 10 (FIG. 11c), 80% 1/20% 2 (FIG. 11d), and 50% 1/50% 2 (FIGS. 11e and 11f).

FIG. 12 shows a graphical representation of release of trypan blue from hydrogels prepared using 100% 1 (hollow circles), 75% 1/25% 2 (diamonds), 50% 1/50% 2 (triangles), 25% 1/75% 2 (squares), and 100% 2 (filled circles) at pH 7.4 over time.

FIG. 13 shows an image of hydrogel strings with encapsulated trypan blue in 1× PBS (FIG. 13a) and a graphical representation of release of trypan blue from hydrogel strings prepared using 100% 1 (circles), 50% 1/50% 2 (triangles), and 100% 2 (squares) at pH 7.4 over time (FIG. 13b).

FIG. 14 shows a graphical representation of release of trypan blue from hydrogel strings prepared using 100% 1 (squares), 50% 1/50% 2 (triangles), and 100% 2 (circles) at pH 5 (FIG. 14a) or pH 9 (FIG. 14b) over time.

FIG. 15 shows a graphical representation of release of a DNA primer from hydrogel strings prepared using 100% 1 (squares), 50% 1/50% 2 (triangles), and 100% 2 (circles) at pH 7.4 over time.

FIG. 16 shows an illustration of the neuron co-culture assays, where primary hippocampal neurons were exposed to hydrogel alone, or hydrogel comprising either 15,000 or 25,000 human amnion epithelial cells encapsulated within the hydrogel (FIG. 16a), and an image of a neuron being studied electrophysiologically using the patch clamp technique (FIG. 16b).

FIG. 17 shows graphical representations of primary hippocampal neuron function as assessed by the electrophysiology patch clamp technique after 4 days of in vitro culture (DIV4) and after 7 days of in vitro culture (DIV7), showing changes in sodium current (in pA/pF) at different stimulus voltages (in mV) for neurons exposed to hydrogel alone (circles), hydrogel comprising 15,000 human amnion epithelial cells encapsulated within the hydrogel (squares) and hydrogel comprising 25,000 human amnion epithelial cells encapsulated within the hydrogel (triangles) (FIG. 17a), changes in neuron synaptic communication when exposed to hydrogel alone or hydrogel comprising either 15,000 or 25,000 human amnion epithelial cells encapsulated within the hydrogel (FIG. 17b) and changes in excitatory post-synaptic potential (EPSPs) amplitude and action potential (AP) frequencies for neurons exposed to hydrogel alone (circles) as compared to neurons exposed to hydrogel comprising either 15,000 (squares) or 25,000 human amnion epithelial cells encapsulated within the hydrogel (triangles) (FIG. 17c).

FIG. 18 shows images of primary hippocampal neuron anatomy as assessed by immunohistochemistry after 4 days of in vitro culture (DIV4) and after 7 days of in vitro culture (DIV7), showing neurons, astroglial support cells with nuclei of all cells, illustrating changes in cultures of neurons exposed to hydrogel comprising either 15,000 (squares) or 25,000 human amnion epithelial cells encapsulated within the hydrogel, suggesting an increase in astrocyte number and synaptic connectivity compared to hydrogel alone.

### DETAILED DESCRIPTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term “about” refers to a quantity, level, value, dimension, size, or amount that varies by as much as 30%, 25%, 20%, 15% or 10% to a reference quantity, level, value, dimension, size, or amount.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The term “hydrophilic” refers to a molecule or portion of a molecule that is attracted to water and other polar solvents. A hydrophilic molecule or portion of a molecule is polar and/or charged or has an ability to form interactions such as hydrogen bonds with water or polar solvents.

The term “hydrophobic” refers to a molecule or portion of a molecule that repels or is repelled by water and other polar solvents. A hydrophobic molecule or portion of a molecule is non-polar, does not bear a charge and is attracted to non-polar solvents.

The term “amphiphilic” refers to molecules having both hydrophilic and hydrophobic regions. The term amphiphilic is synonymous with “amphipathic” and these terms may be used interchangeably.

As used herein, the term “amino acid” refers to an α-amino acid or a β-amino acid and may be a L-or D-isomer.

As used herein, the term “β-amino acid” refers to an amino acid that has two (2) carbon atoms separating a carboxyl terminus (C-terminus) and an amino terminus (N-terminus). In the context of the present invention, β-amino acids with a specific side chain can exist as the R or S enantiomers at the β(C3) carbon (i.e. a β³-amino acid), resulting in a total of 2 possible isomers for any given side chain. The side chains may be the same as those of naturally occurring α-amino acids or may be the side chains of non-naturally occurring amino acids.

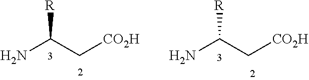

Suitable side chains for the β-amino acid include —C₁₋₆alkyl, —(CH₂)ₙCORₐ, —(CH₂)ₙR_{b}, —PO₃H, —(CH₂)ₙheterocyclyl or —(CH₂)ₙaryl where n is an integer selected from 1 to 8, Rₐ is —OH, —NH₂, —NHC₁₋₃alkyl, —OC₁₋₃alkyl or —C₁₋₃alkyl and R_{b} is —OH, —SH, —SC₁₋₃alkyl —OC₁₋₃alkyl, —C₃₋₁₂cycloalkyl, —NH₂, —NHC₁₋₃alkyl or —NHC(C═NH)NH₂ and where each alkyl, cycloalkyl, aryl or heterocyclyl group may be substituted with one or more groups selected from —OH, —NH₂, —NHC₁—C₃alkyl, —OC₁₋₃alkyl, —SH, —SC₁₋₃alkyl, —COH, —CO₂C₁₋₃alkyl, —CONH₂ or —CONHC₁₋₃alkyl.

Suitable derivatives of β-amino acids include salts and derivatives where functional groups protected by suitable protecting groups.

As used herein, the term “α-amino acid” refers to an amino acid that has a single carbon atom (the α-carbon atom) separating a carboxyl terminus (C-terminus) and an amino terminus (N-terminus). The α-amino acids may have a naturally occurring side chain a non-naturally occurring amino acid side chain.

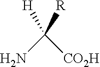

The α-amino acid may be have a side chain selected from —C₁₋₆alkyl, —(CH₂)ₙCORₐ, —(CH₂)ₙR_{b}, —PO₃H, —(CH₂)ₙ heterocyclyl or —(CH₂)ₙaryl where n is an integer selected from 1 to 8, Rₐ is —OH, —NH₂, —NHC₁₋₃alkyl, —OC₁₋₃alkyl or —C₁₋₃alkyl and R_{b} is —OH, —SH, —SC₁₋₃alkyl —OC₁₋₃alkyl, —C₃₋₁₂cycloalkyl, —NH₂, —NHC₁₋₃alkyl or —NHC(C═NH)NH₂ and where each alkyl, cycloalkyl, aryl or heterocyclyl group may be substituted with one or more groups selected from —OH, —NH₂, —NHC₁-C₃alkyl, —OC₁₋₃alkyl, —SH, —SC₁₋₃alkyl, —CO₂H, —CO₂C₁₋₃alkyl, —CONH₂ or —CONHC₁₋₃alkyl.

Suitable derivatives of α-amino acids include salts and derivatives where functional groups protected by suitable protecting groups

The term “naturally occurring amino acid side chain” as used herein refers to an amino acid side chain that occurs in the naturally occurring L-α-amino acids. Examples of naturally occurring amino acid side chains are presented below in Table 1.

TABLE 1

| | Three-letter | |
|---|---|---|
| Amino Acid | Abbreviation | Structure of side chain (R) |
| Alanine | Ala | —CH₃ |
| Arginine | Arg | —(CH₂)₃NHC(═N)NH₂ |
| Asparagine | Asn | —CH₂CONH₂ |
| Aspartic acid | Asp | —CH₂CO₂H |
| Cysteine | Cys | —CH₂SH |
| Glutamine | Gln | —(CH₂)₂CONH₂ |
| Glutamic acid | Glu | —(CH₂)₂CO₂H |
| Glycine | Gly | —H |
| Histidine | His | —CH₂(4-imidazolyl) |
| Isoleucine | Ile | —CH(CH₃)CH₂CH₃ |
| Leucine | Leu | —CH₂CH(CH₃)₂ |
| Lysine | Lys | —(CH₂)₄NH₂ |
| Methionine | Met | —(CH₂)₂SCH₃ |
| Phenylalanine | Phe | —CH₂Ph |
| Proline | Pro | see formula below for |
| | | α-amino acid structure |
| Serine | Ser | —CH₂OH |
| Threonine | Thr | —CH(CH₃)OH |
| Tryptophan | Trp | —CH₂(3-indolyl) |
| Tyrosine | Tyr | —CH₂(4-hydroxyphenyl) |
| Valine | Val | —CH(CH₃)₂ |

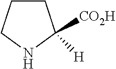

The term “non-naturally occurring amino acid side chain” as used herein refers to an amino acid side chain that does not occur in the naturally occurring L-α-amino acids. Examples of non-natural amino acid side chains and derivatives include, but are not limited to, the side chains of norleucine, norvaline, 4-aminobutyric acid, 2-aminoisobutyric acid, cyclohexylalanine, cyclopentylalanine, naphthylalanine, phenylglycine, t-butylglycine, ornithine, and/or D-isomers of amino acids.

The term “hydrophobic amino acid side chain” as used herein refers to an amino acid side chain which is non-polar. Examples include, but are not limited to, the side chains of alanine, valine, leucine, isoleucine, proline, methionine, phenylalanine, tryptophan, aminoisobutyric acid, cyclohexylalanine, cyclopentylalanine, norleucine, norvaline, tert-butylglycine and ethylglycine, especially alanine, valine, leucine, isoleucine, proline, methionine, phenylalanine, tryptophan and aminoisobutyric acid.

The term “hydrophilic amino acid side chain” as used herein refers to an amino acid side chain which is polar or charged. Examples include, but are not limited to, the side chains of glycine, serine, threonine, cysteine, tyrosine, asparagine, glutamine, aspartic acid, glutamic acid, lysine, arginine, histidine and ornithine.

As used herein, the term “positively charged amino acid side chain” refers to an amino acid side chain capable of bearing a positive charge. Examples include, but are not limited to, the side chains of lysine, arginine, histidine and ornithine.

As used herein, the term “negatively charged amino acid side chain” refers to an amino acid side chain capable of bearing a negative charge. Examples include, but are not limited to, the side chains of aspartic acid and glutamic acid.

As used herein, the term “polar amino acid side chain” refers to an amino acid side chain that has a dipole moment. Examples include, but are not limited to, the sidechains of glycine, serine, threonine, cysteine, tyrosine, asparagine and glutamine.

Those skilled in the art will appreciate that a peptide represents a series of two or more amino acids linked through a covalent bond formed between the carboxyl group of one amino acid and the amino group of another amino acid (i.e. the so-called peptide bond). Accordingly, a “β-peptide” refers to a peptide that comprises two or more sequential β-amino acids, and an “α-peptide” refers to a peptide that comprises two or more sequential α-amino acids.

The term “alkyl” as used herein refers to straight chain or branched hydrocarbon groups. Where appropriate, the alkyl group may have a specified number of carbon atoms, for example, C₁₋₆alkyl which includes alkyl groups having 1, 2, 3, 4, 5 or 6 carbon atoms in a linear or branched arrangement. Examples of suitable alkyl groups include, but are not limited to, methyl, ethyl, n-propyl, i-propyl, n-butyl, i-butyl, t-butyl, n-pentyl, 2-methylbutyl, 3-methylbutyl, 4-methylbutyl, n-hexyl, 2-methylpentyl, 3-methylpentyl, 4-methylpentyl, 5-methylpentyl, 2-ethylbutyl, 3-ethylbutyl, heptyl, octyl, nonyl, decyl, undecyl, dodecyl, tridecyl, tetradecyl, pentadecyl, hexadecyl, heptadecyl, octadecyl, nonadecyl and eicosanyl.

The term “alkenyl” as used herein refers to a straight-chain or branched hydrocarbon group having one or more double bonds between carbon atoms and having 2 to 10 carbon atoms. Where appropriate, the alkenyl group may have a specified number of carbon atoms. For example, C₂-C₆ as in “C₂-C₆alkenyl” includes groups having 2, 3, 4, 5 or 6 carbon atoms in a linear or branched arrangement. Examples of suitable alkenyl groups include, but are not limited to, ethenyl, propenyl, isopropenyl, butenyl, butadienyl, pentenyl, pentadienyl, hexenyl, hexadienyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl and eicosenyl.

As used herein, the term “alkynyl” refers to a straight-chain or branched hydrocarbon group having one or more triple bonds and having 2 to 10 carbon atoms. Where appropriate, the alkynyl group may have a specified number of carbon atoms. For example, C₂-C₆ as in “C₂-C₆alkynyl” includes groups having 2, 3, 4, 5 or 6 carbon atoms in a linear or branched arrangement. Examples of suitable alkynyl groups include, but are not limited to ethynyl, propynyl, butynyl, pentynyl, hexynyl, heptynyl, octynyl, nonynyl, decynyl, undecynyl, dodecynyl, tridecynyl, tetradecynyl, pentadecynyl, hexadecynyl, heptadecynyl, octadecynyl, nonadecynyl and eicosynyl.

The term “cycloalkyl” as used herein, refers to cyclic hydrocarbon groups that may include one or more double bonds but are not aromatic. Suitable cycloalkyl groups include, but are not limited to, cyclopropyl, cyclobutyl, cyclopentyl, cyclohexyl, cyclohexenyl, cyclohexadienyl, cycloheptyl, cyclooctyl, cyclononyl, cyclodecyl, cycloundecyl and cyclododecyl.

The term “aryl” as used herein, refers to C₆-C₁₀ aromatic hydrocarbon groups, for example phenyl and naphthyl.

The term “heterocyclyl” as used herein refers to a cyclic hydrocarbon in which one to four carbon atoms have been replaced by heteroatoms independently selected from the group consisting of N, N(R), S, S(O), S(O)₂ and O. A heterocyclic ring may be saturated or unsaturated but not aromatic. Examples of suitable heterocyclyl groups include azetidine, tetrahydrofuranyl, tetrahydrothiophenyl, pyrrolidinyl, 2-oxopyrrolidinyl, pyrrolinyl, pyranyl, dioxolanyl, piperidinyl, 2-oxopiperidinyl, pyrazolinyl, imidazolinyl, thiazolinyl, dithiolyl, oxathiolyl, dioxanyl, dioxinyl, dioxazolyl, oxathiozolyl, oxazolonyl, piperazinyl, morpholino, thiomorpholinyl, 3-oxomorpholinyl, dithianyl, trithianyl and oxazinyl.

The term “heteroaryl” as used herein, represents a stable monocyclic, bicyclic or tricyclic ring of up to 7 atoms in each ring, wherein at least one ring is aromatic and at least one ring contains from 1 to 4 heteroatoms selected from the group consisting of O, N and S. Heteroaryl groups within the scope of this definition include, but are not limited to, acridinyl, carbazolyl, cinnolinyl, quinoxalinyl, quinazolinyl, pyrazolyl, indolyl, isoindolyl, 1H,3H-1-oxoisoindolyl, benzotriazolyl, furanyl, thienyl, thiophenyl, benzothienyl, benzofuranyl, benzodioxane, benzodioxin, quinolinyl, isoquinolinyl, oxazolyl, isoxazolyl, imidazolyl, pyrazinyl, pyridazinyl, pyridinyl, pyrimidinyl, pyrrolyl, tetrahydroquinolinyl, thiazolyl, isothiazolyl, 1,2,3-triazolyl, 1,2,4-triazolyl, 1,2,4-oxadiazolyl, 1,2,4-thiadiazolyl, 1,3,5-triazinyl, 1,2,4-triazinyl, 1,2,4,5-tetrazinyl and tetrazolyl.

The term “N-terminal capping group” as used herein is any group that blocks the reactivity of the N-terminal amino group of an amino acid or peptide. Suitable examples include acyl groups such as acetyl (ethanoyl), propanoyl, butanoyl, pentanoyl and hexanoyl.

The term “C-terminal capping group” as used herein is any suitable group that blocks the reactivity of the C-terminal carboxyl group of an amino acid or peptide. Suitable examples include amino groups thereby forming an amide. Examples include —NH₂, —NH(alkyl) and —N(alkyl)₂.

As used herein, the term “hydrogel” refers to a gel formed by a polymer network in which the swelling agent is water.

### 2. β-Peptides

Compounds useful in the present invention are β-peptides of formula (I):

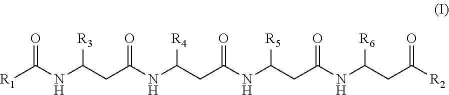

wherein
R₁ is selected from —C₁₋₂₀alkyl, —C₂₋₂₀alkenyl, —C₂₋₂₀alkynyl, —OC₁₋₂₀alkyl, —OC₂₋₂₀alkenyl and —OC₂₋₂₀alkynyl;
R₂ is selected from —H, —OR₁₀ₐ, —SR₁₀ₐ, —N(R₁₁ₐ)₂, —NH(C═NH)NH₂, cycloalkyl, aryl, heterocyclyl, and heteroaryl, where each cycloalkyl, aryl, heterocyclyl, and heteroaryl is unsubstituted or is substituted with one or more groups selected from —OH, —NH₂, —NHC₁₋₃alkyl, —OC₁₋₃alkyl, —SH, —SC₁₋₃alkyl, —CO₂H, —CO₂C₁₋₃alkyl, —CONH₂ and —CONHC₁₋₃alkyl, or R₂ has the following structure (A):

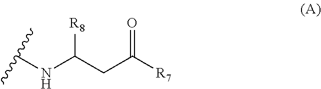

R₃, R₄, R₅, R₆ and R₈ are each independently selected from —H, —R₉, —C₁₋₆alkylR₉, —C₂₋₆alkenylR₉ and —C₂₋₆alkynylR₉;
or R₂ and R₆ together form a heterocyclic ring;
R₇ is selected from —H, —OR₁₀ₐ, —SR₁₀ₐ, —N(R₁₁ₐ)₂, —NH(C═NH)NH₂, cycloalkyl, aryl, heterocyclyl, and heteroaryl, where each cycloalkyl, aryl, heterocyclyl, and heteroaryl is unsubstituted or is substituted with one or more groups selected from —OH, —NH₂, —NHC₁₋₃alkyl, —OC₁₋₃alkyl, —SH, —SC₁₋₃alkyl, —CO₂H, —CO₂C₁₋₃alkyl, —CONH₂ and —CONHC₁₋₃alkyl; or R₇ and R₈ together form a heterocyclic ring;
R₉ is selected from —H, —OR₁₀, —SR₁₀, —N(R₁₁)₂, —C(O)R₁₂, —NH(C═NH)NH₂, cycloalkyl, aryl, heterocyclyl, and heteroaryl, where each cycloalkyl, aryl, heterocyclyl, and heteroaryl is unsubstituted or is substituted with one or more groups selected from —OH, —NH₂, —NHC₁₋₃alkyl, —OC₁₋₃alkyl, —SH, —SC₁₋₃alkyl, —CO₂H, —CO₂C₁₋₃alkyl, —CONH₂ and —CONHC₁₋₃alkyl;
R₁₀ is selected from —H, —C₁₋₆alkyl, —C₂₋₆alkenyl, —C₂₋₆alkynyl, —C(O)C₁₋₂₀alkyl, —C(O)C₁₋₂₀alkenyl, —C(O)C₁₋₂₀alkynyl, —C(O)C₁₋₆alkylR₁₄, an α-peptide having 1 to 10 α-amino acid residues and an imaging agent;
R₁₀ₐ is selected from —H, —C₁₋₆alkyl, —C₂₋₆alkenyl, —C₂₋₆alkynyl, —C(O)C₁₋₆alkyl, —C(O)C₂₋₆alkenyl, and —C(O)C₂₋₆alkynyl;
each R¹¹ is independently selected from —H, —C₁₋₆alkyl, —C₁₋₆alkylN(R₁₃)₂, —C₁₋₆alkylNR₁₃R₁₄, —C₂₋₆alkenyl, —C₂₋₆alkynyl, —C(O)C₁₋₂₀alkyl, —C(O)C₁₋₂₀alkenyl, —C(O)C₁₋₂₀alkynyl, —C(O)C₁₋₆alkylR₁₄, an α-peptide having 1 to 10 α-amino acid residues and an imaging agent;
each R₁₁ₐ is independently selected from —H, —C₁₋₆alkyl, —C₂₋₆alkenyl, —C₂₋₆alkynyl, —C(O)C₁₋₆alkyl, —C(O)C₂₋₆alkenyl, —C(O)C₂₋₆alkynyl;
R₁₂ is selected from —OH, —OR₁₀ and —N(R₁₁)₂;
each R₁₃ is independently selected from —H and —C₁₋₆alkyl;
R₁₄ is an α-peptide having 1 to 10 α-amino acid residues or an imaging agent;
or a pharmaceutically acceptable salt thereof;
and wherein one of R₁, R₂, R₃, R₄, R₅, R₆ and R₈ comprise a C₈₋₂₀alkyl group, a C₈₋₂₀alkenyl group or a C₈₋₂₀alkynyl group.

In some embodiments of formula (I), R₁ is C₁₋₂₀alkyl. In some embodiments of formula (I), R₁ is C₁₋₄ alkyl. In some embodiments of formula (I), R₁ is methyl.

In some embodiments of formula (I), R₂ is selected from the group consisting of —H, —OR₁₀ₐ, —SR₁₀ₐ and —N(R₁₁ₐ)₂. In some embodiments of formula (I), R₂ is H.

In some embodiments of formula (I), R₂ and R₆ together form a heterocyclic ring. In some embodiments, of formula (I), R₂ and R₆ together form a lactone. In some embodiments, the β-peptide of formula (I) has the formula:

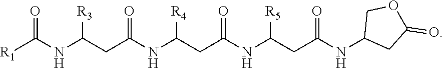

In some embodiments of formula (I), R₂ has the structure (A):

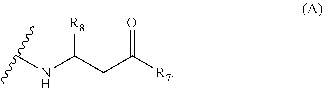

In some embodiments of formula (I), R₈ and R₇ together form a heterocyclic ring. In some embodiments, of formula (I), R₈ and R₇ together form a lactone. In some embodiments, the β-peptide of formula (I) has the formula:

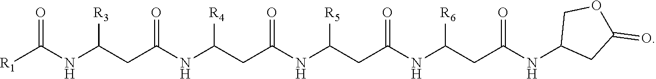

In some embodiments of formula (I), R₃ is —C₁₋₆alkylR₉. In some embodiments of formula (I), R₃ is —C₁₋₆alkylR₉, wherein R₉ is —N(R₁₀)₂, wherein one R₁₀ is H, and the other R₁₀ is —C(O)C₁₋₂₀alkyl or —C(O)C₂₋₂₀alkenyl.

In some embodiments of formula (I), R₄ is —C₁₋₆alkylR₉. In some embodiments of formula (I), R₄ is —C₁₋₆alkylR₉, wherein R₉ is H or OH.

In some embodiments of formula (I), R₅ is —R₉ or —C₁₋₆alkylR₉.

In some embodiments of formula (I), R₅ is R₉, wherein R₉ is —C(O)R₁₂, wherein R₁₂ is —N(R₁₁)₂, wherein one R₁₁ is H, and the other R₁₁ is —C₁₋₆alkylNR₁₃R₁₄, wherein R₁₃ is H, and R₁₄ is selected from the group consisting of an α-peptide having 1 to 10 α-amino acid residues and an imaging agent. In some embodiments, R₁₂ is

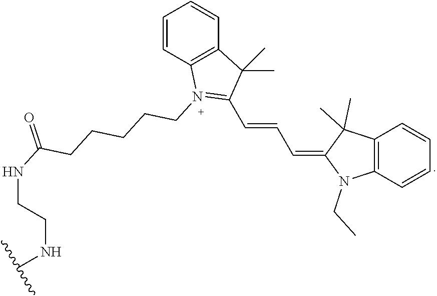

In some embodiments, R₁₂ is

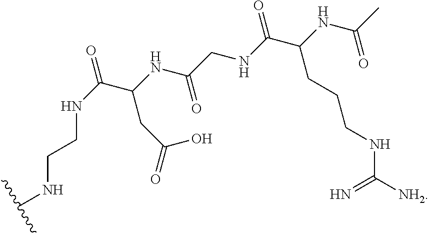 [SMILES: *NCCNC(=O)C(CC(=O)O)NC(=O)CNC(=O)C(CCCNC(=N)N)NC(C)=O]

In some embodiments, R₁₂ is

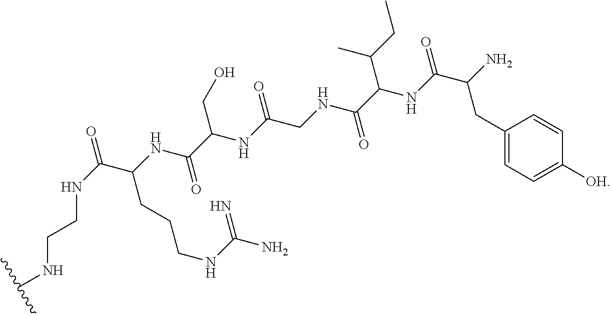 [SMILES: *NCCNC(=O)C(CCCNC(=N)N)NC(=O)C(CO)NC(=O)CNC(=O)C(NC(=O)C(N)Cc1ccc(O)cc1)C(C)CC]

In some embodiments, R₁₂ is

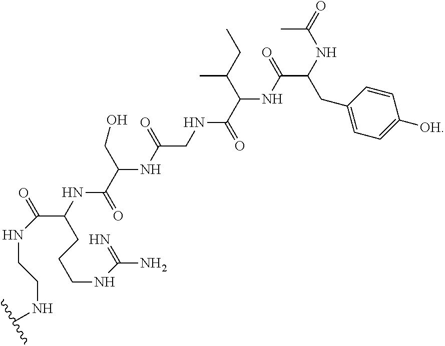 [SMILES: *NCCNC(=O)C(CCCNC(=N)N)NC(=O)C(CO)NC(=O)CNC(=O)C(NC(=O)C(Cc1ccc(O)cc1)NC(C)=O)C(C)CC]

In some embodiments of formula (I), R₆ is —C₁₋₆alkylR₉. In some embodiments of formula (I), R₆ is —C₁₋₆alkylR₉, wherein R₉ is —NH₂.

In some embodiments of formula (I):
R₁ is selected from —C₁₋₂₀alkyl, —C₂₋₂₀alkenyl, —OC₁₋₂₀alkyl and —OC₂₋₂₀alkenyl;
R₂ is —OH, or R₂ has the following structure (A):

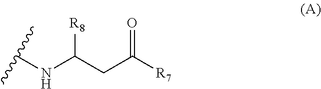

R₃, R₄, R₅, R₆ and R₈ are each independently selected from —H, —R₉, and —C₁₋₆alkylR₉;
or R₂ and R₆ together form a heterocyclic ring;
R₇ is —OH;
or R₇ and R₈ together form a heterocyclic ring;
R₉ is selected from —H, —OR₁₀, —SR₁₀, —N(R₁₀)₂, —C(O)R₁₂, —NH(C═NH)NH₂, aryl, and heteroaryl, where each aryl and heteroaryl is unsubstituted or substituted with one or more groups selected from —OH, —NH₂, —NHC₁₋₃alkyl, —OC₁₋₃alkyl, —SH, —SC₁₋₃alkyl, —CO₂H, —CO₂C₁₋₃alkyl, —CONH₂ or —CONHC₁₋₃alkyl;
R₁₀ is selected from —H, —C₁₋₆alkyl, —C(O)C₁₋₂₀alkyl, —C(O)C₁₋₂₀alkenyl, —C(O)C₁₋₆alkylR₁₄, an α-peptide having 1 to 10 α-amino acid residues and an imaging agent;
each R₁₁ is independently selected from —H, —C₁₋₆alkyl, —C₁₋₆alkylN(R₁₃)₂, —C₁₋₆alkylNR₁₃R₁₄, —C₂₋₆alkenyl, —C₂₋₆alkynyl, —C(O)C_{1- 20}alkyl, —C(O)C₁₋₂₀alkenyl, —C(O)C₁₋₆alkylR₁₄, an α-peptide having 1 to 10 α-amino acid residues and an imaging agent;
R₁₂ is selected from —OH, —OR₁₀ and —N(R₁₁)₂;
each R₁₃ is independently selected from —H and —C₁₋₆alkyl;
R₁₄ is an α-peptide having 1 to 10 α-amino acid residues or an imaging agent;
or a pharmaceutically acceptable salt thereof;
and wherein one of R₁, R₂, R₃, R₄, R₅, R₆ and R₈ comprise a C₈₋₂₀alkyl or a C₈₋₂₀alkenyl group.

In particular embodiments of formula (I), one or more of the following applies:
R₁ is selected from —C₁₋₂₀alkyl, especially —C₁₋₅alkyl or —C₁₁₋₁₅alkyl, more especially methyl or —C₁₁₋₁₅alkyl, and —OC₁₋₂₀alkyl, especially —OC₁₋₅alkyl or —OC₁₁₋₁₅alkyl;
R₂ is —OH;
R₃, R₄, R₅ and R₆ are each independently selected from —H, —R₉, and —C₁₋₆alkylR₉;
or R₂ and R₆ together form a heterocyclic ring, especially a lactone;
R₉ is selected from —H, —OR₁₀, —SR₁₀, —N(R₁₁)₂, —C(O)R₁₂, —NH(C═NH)NH₂, unsubstituted aryl, especially phenyl, unsubstituted heteroaryl, especially indoyl, and aryl or heteroaryl substituted with one or more groups selected from —OH, —NH₂, —CO₂H, —CO₂C₁₋₃alkyl or —CONH₂, especially 4-hydroxyphenyl;
R₁₀ is selected from —C₁₋₆alkyl;
each R₁₁ is independently selected from —H, —C₁₋₆alkylNR₁₃R₁₄, and —C(O)C₁₋₂₀alkyl, especially —C(O)C₁₁₋₁₅alkyl;
R₁₂ is selected from —OH and —N(R₁₁)₂;
R₁₃ is —H;
R₁₄ is an α-peptide having 1 to 10 α-amino acid residues or an imaging agent;
or a pharmaceutically acceptable salt thereof;
and wherein one of R₁, R₂, R₃, R₄, R₅ and R₆ comprise a C₈₋₂₀alkyl group, especially R₃ comprises the C₈₋₂₀alkyl group, more especially R₃ is —CH₂NHC(O)C₈₋₂₀alkyl.

In some embodiments, one or more of R₃, R₄, R₅ and R₆ (and R₈, when present) corresponds to an amino acid side chain, where each amino acid side chain may be the same or different. In these embodiments, the amino acid side chain cannot be a proline side chain and is preferably not a cysteine or histidine side chain. In particular embodiments, one or more of the following applies:
one or more of R₃, R₄, R₅ and R₆ (and R₈, when present) corresponds to a hydrophilic amino acid side chain, where each hydrophilic amino acid side chain may be the same or different;
one or more of R₄, R₅ and R₆ corresponds to a hydrophilic amino acid side chain, where each hydrophilic amino acid side chain may be the same or different;
one or more of R₄, R₅ and R₆, especially R₆, corresponds to a positively charged amino acid side chain, especially lysine, where each positively charged amino acid side chain may be the same or different;
one or more of R₄, R₅ and R₆ corresponds to a polar amino acid side chain, especially serine, where each polar amino acid side chain may be the same or different.
one or more of R₃, R₄, R₅ and R₆ corresponds to a hydrophobic amino acid side chain, where each hydrophobic amino acid side chain may be the same or different;
one or more of R₄, R₅ and R₆, especially R₄, corresponds to a hydrophobic amino acid side chain, especially alanine, where each hydrophobic amino acid side chain may be the same or different.

In some embodiments, one or more of the following applies, especially both:
one of R₄, R₅ and R₆, especially R₆, corresponds to a positively charged amino acid side chain, especially lysine, where each positively charged amino acid side chain may be the same or different;
one of R₄, R₅ and R₆ corresponds to a polar amino acid side chain, especially serine, where each polar amino acid side chain may be the same or different.

The β-peptide of formula (I) may contain an α-peptide having 1 to 10 α-amino acid residues. The α-peptide may be conjugated by its C-terminus or N-terminus, especially by its C-terminus, to R₃, R₄, R₅ or R₆ (or R₈, when present), especially R₅. The non-conjugated end of the α-peptide may have a C-terminal capping group or an N-terminal capping group, especially an acetyl group. In some embodiments, the α-peptide is a cell adhesion motif. The cell adhesion motif may be any suitable motif that can be recognised by a cell and mediate cell attachment. Suitable motifs include integrin-based and lamanin-based cell adhesion motifs. In some embodiments, the α-peptide is a cell adhesion motif selected from RGD, YIGSR (SEQ ID NO: 1), IKVAV (SEQ ID NO: 2) and SIKVAV (SEQ ID NO: 3), especially RGD or YIGSR, more especially RGD.

The β-peptide of formula (I) may contain an imaging agent. The imaging agent may be any suitable agent that can allow for visualisation of the β-peptide via an appropriate imaging technique. The imaging agent may be conjugated directly to R₃, R₄, R₅ or R₆ (or R₈, when present), especially R₅, by any suitable bond, for example an amide bond. Suitable imaging agents include chromophores and fluorophores. In some embodiments, the imaging agent is a fluorophore. The fluorophore may be any suitable molecule having an ability to absorb light at a particular wavelength and re-emit that light at a higher wavelength. In some embodiments, the fluorophore is a reactive dye. In some embodiments, the dye has a maximum excitation wavelength within the range of 400 to 800 nm, especially 550 to 700 nm. In some embodiments, the dye has a maximum emission wavelength within the range of 400 to 800 nm, especially 560 to 710 nm. Suitable dyes include cyanine dyes, especially closed chain cyanines such as Quasar® dyes.

In particular embodiments, the compounds of the invention are selected from the following:

| No. | Structure |
|---|---|
| 1 | 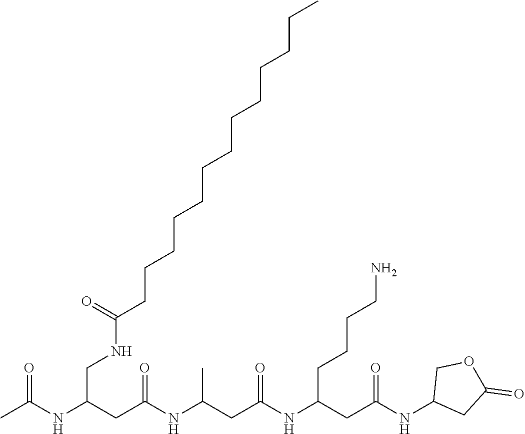 [SMILES: CCCCCCCCCCCCCC(=O)NCC(CC(=O)NC(C)CC(=O)NC(CCCCN)CC(=O)NC1COC(=O)C1)NC(C)=O] |
| 2 | 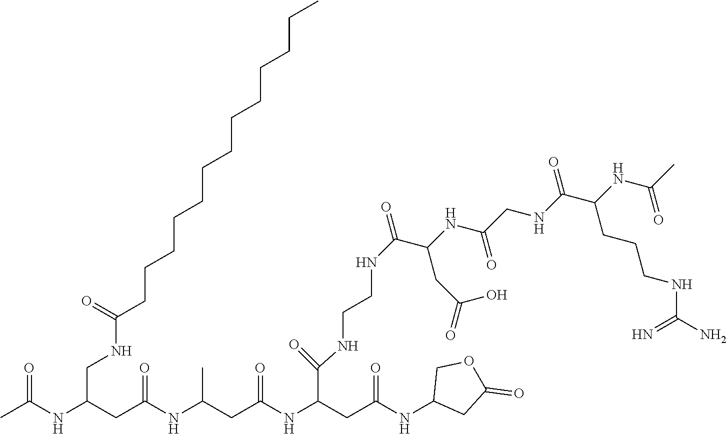 [SMILES: CCCCCCCCCCCCCC(=O)NCC(CC(=O)NC(C)CC(=O)NC(CC(=O)NC1COC(=O)C1)C(=O)NCCNC(=O)C(CC(=O)O)NC(=O)CNC(=O)C(CCCNC(=N)N)NC(C)=O)NC(C)=O] |
| 3 | 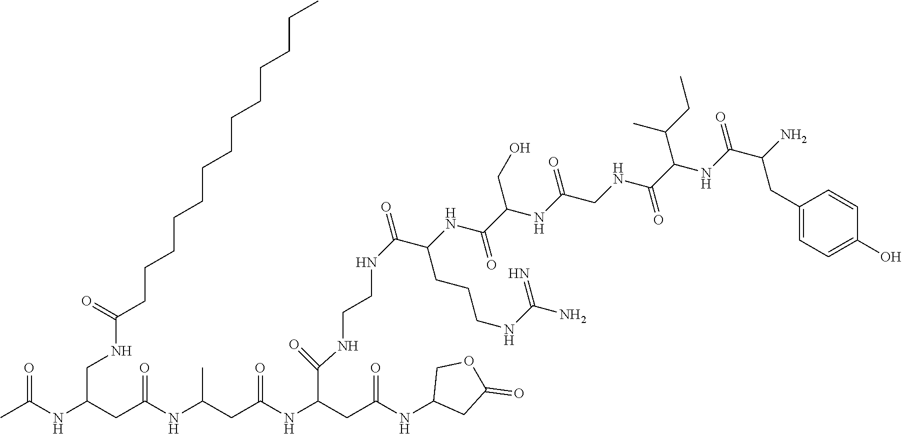 [SMILES: CCCCCCCCCCCCCC(=O)NCC(CC(=O)NC(C)CC(=O)NC(CC(=O)NC1COC(=O)C1)C(=O)NCCNC(=O)C(CCCNC(=N)N)NC(=O)C(CO)NC(=O)CNC(=O)C(NC(=O)C(N)Cc1ccc(O)cc1)C(C)CC)NC(C)=O] |
| | (SEQ ID NO: 4) |
| 4 | 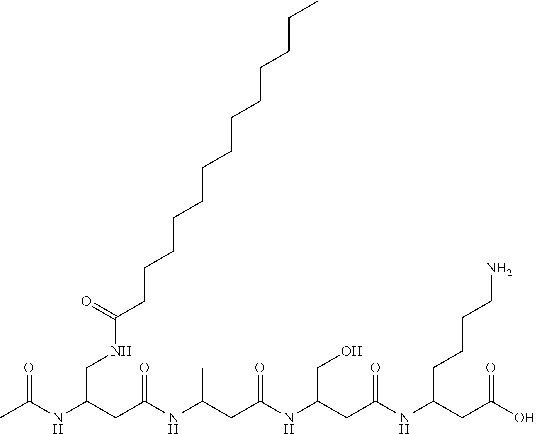 [SMILES: CCCCCCCCCCCCCC(=O)NCC(CC(=O)NC(C)CC(=O)NC(CO)CC(=O)NC(CCCCN)CC(=O)O)NC(C)=O] |
| 5 | 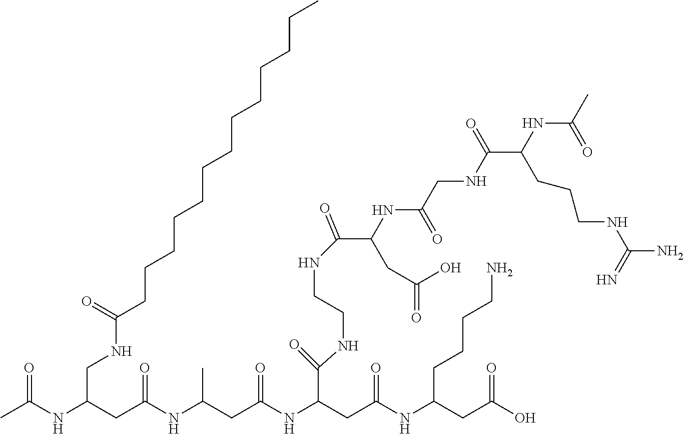 [SMILES: CCCCCCCCCCCCCC(=O)NCC(CC(=O)NC(C)CC(=O)NC(CC(=O)NC(CCCCN)CC(=O)O)C(=O)NCCNC(=O)C(CC(=O)O)NC(=O)CNC(=O)C(CCCNC(=N)N)NC(C)=O)NC(C)=O] |
| 6 | 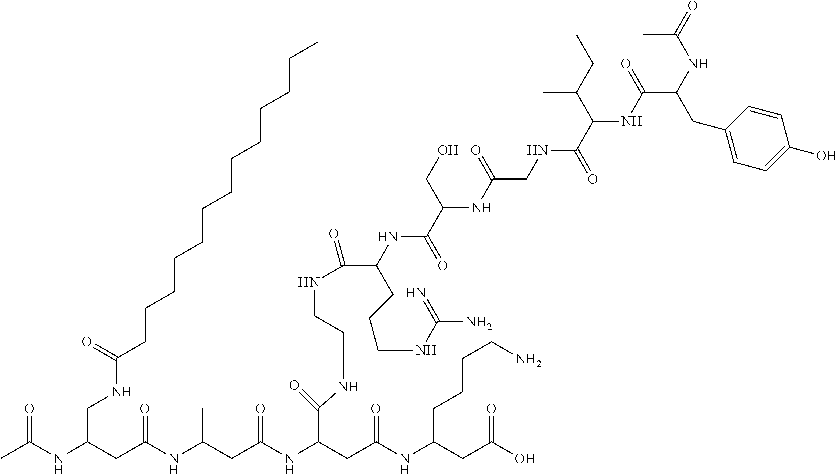 [SMILES: CCCCCCCCCCCCCC(=O)NCC(CC(=O)NC(C)CC(=O)NC(CC(=O)NC(CCCCN)CC(=O)O)C(=O)NCCNC(=O)C(CCCNC(=N)N)NC(=O)C(CO)NC(=O)CNC(=O)C(NC(=O)C(Cc1ccc(O)cc1)NC(C)=O)C(C)CC)NC(C)=O] |
| | (SEQ ID NO: 5) |
| 7 | 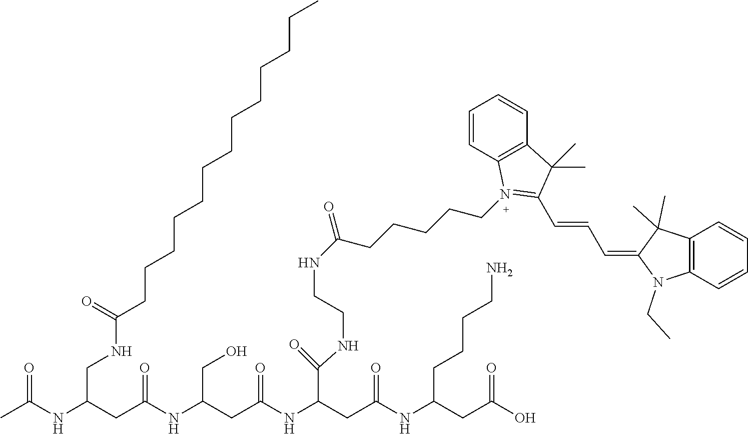 |

The β-peptides of the invention may be synthesised according to known methods, including solid-phase and solution-phase peptide synthesis. Advantageously, the β-peptides of the invention may be synthesised by solid-phase peptide synthesis using suitable solid supports, protecting groups and coupling reagents, which allows for facile and rapid synthesis of the β-peptides. For example, the β-peptides may be prepared using standard Fmoc chemistry on Wang resin. The resin may be swollen in a suitable solvent such as dimethyl formamide. A first Fmoc-protected β-amino acid may then be activated and coupled to the resin using suitable activating and coupling reagents as known in the art. The Fmoc protecting group of the first β-amino acid may then be deprotected and then coupled with a second β-amino acid. These coupling cycles may be repeated until the complete β-peptide is assembled. The cycles may include a capping step that blocks the ends of unreacted amino acids from reacting. The cycles may further include functionalising a β-amino acid side chain using methods known in the art, for example with by reacting a functional group of the β-amino acid side chain with a fatty acid, an α-peptide having 1 to 10 α-amino acid residues or an imaging agent such that the fatty acid, α-peptide or imaging agent is conjugated to the β-amino acid side chain. Once the complete β-peptide is assembled, the β-peptide may then be cleaved from the resin using a suitable cleavage solution, where the cleavage solution may also simultaneously remove any remaining protecting groups on the β-peptide that are susceptible to deprotection by the cleavage solution.

The β-peptides of the invention may also be synthesised using methods analogous to those described in Example 1.

The β-peptides according to the present invention have an acylated N-terminus and include a C₈₋₂₀alkyl group, a C₈₋₂₀alkenyl group or a C₈₋₂₀alkynyl group within their structure. As shown in the examples, the β-peptides can spontaneously self-assemble in aqueous solution to form fibers. Without wishing to be bound by theory, the present inventors hypothesise that the β-peptides self-assemble into helical structures as a result of donor-acceptor hydrogen bonding interactions between the N-terminal acyl carbonyl and backbone peptide bonds of the β-peptides. Hydrophobic interactions between the C₈₋₂₀alkyl, C₈₋₂₀alkenyl or C₈₋₂₀alkynyl groups of the β-peptides then drives the formation of fibres in a similar manner to peptide amphiphiles. Provided that the features of an acylated N-terminus and a C₈₋₂₀alkyl, C₈₋₂₀alkenyl or C₈₋₂₀alkynyl group are present, self-assembly of the (β-peptides can occur regardless of monomer sequence.

As discussed below, the β-peptides of the invention are capable of forming hydrogels in aqueous solution. In order to form hydrogels, the β-peptides of formula (I) should be suitably soluble or partially soluble in aqueous solution. The presence of the C₈₋₂₀alkyl, C₈₋₂₀alkenyl or C₈₋₂₀alkynyl group increases the hydrophobicity of the β-peptide and may decrease the water solubility of the β-peptides. To counteract this, polar and/or charged groups can be included in the β-peptide structure to increase hydrophilicity (and therefore water solubility).

### 3. β-Peptide Hydrogels

The β-peptides of formula (I) may form hydrogels in aqueous solution. As described above, the β-peptides can spontaneously self-assemble into fibres in solution. This leads to the formation of fibrillar networks that absorbs water, resulting in the formation of a hydrogel.

Accordingly, the present invention provides a hydrogel comprising the β-peptides of the present invention. In particular, the present invention provides a hydrogel comprising one or more β-peptides of formula (I):

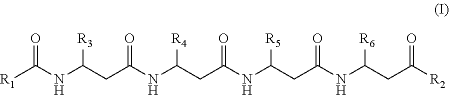

wherein
R₁ is selected from —C₁₋₂₀alkyl, —C₂₋₂₀alkenyl, —C₂₋₂₀alkynyl, —OC₁₋₂₀alkyl, —OC₂₋₂₀alkenyl and —OC₂₋₂₀alkynyl;
R₂ is selected from —H, —OR₁₀ₐ, —SR₁₀ₐ, —N(R₁₁ₐ)₂, —NH(C═NH)NH₂, cycloalkyl, aryl, heterocyclyl, and heteroaryl, where each cycloalkyl, aryl, heterocyclyl, and heteroaryl is unsubstituted or is substituted with one or more groups selected from —OH, —NH₂, —NHC₁₋₃alkyl, —OC₁₋₃alkyl, —SH, —SC₁₋₃alkyl, —CO₂H, —CO₂C₁₋₃alkyl, —CONH₂ and —CONHC₁₋₃alkyl, or R₂ has the following structure (A):

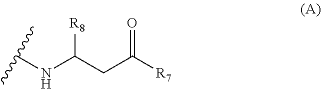

R₃, R₄, R₅, R₆ and R₈ are each independently selected from —H, —R₉, —Cₗ₋₆alkylR₉, —C₂₋₆alkenylR₉ and —C₂₋₆alkynylR₉;
or R₂ and R₆ together form a heterocyclic ring; R₇ is selected from —H, —OR₁₀ₐ, —SR₁₀ₐ, —N(R₁₁ₐ)₂, —NH(C═NH)NH₂, cycloalkyl, aryl, heterocyclyl, and heteroaryl, where each cycloalkyl, aryl, heterocyclyl, and heteroaryl is unsubstituted or is substituted with one or more groups selected from —OH, —NH₂, —NHC₁₋₃alkyl, —OC₁₋₃alkyl, —SH, —SC₁₋₃alkyl, —CO₂H, —CO₂C₁₋₃alkyl, —CONH₂ and —CONHC₁₋₃alkyl; or R₇ and R₈ together form a heterocyclic ring;
R₉ is selected from —H, —OR₁₀, —SR₁₀, —N(R₁₁)₂, —C(O)R₁₂, —NH(C═NH)NH₂, cycloalkyl, aryl, heterocyclyl, and heteroaryl, where each cycloalkyl, aryl, heterocyclyl, and heteroaryl is unsubstituted or is substituted with one or more groups selected from —OH, —NH₂, —NHC₁₋₃alkyl, —OC₁₋₃alkyl, —SH, —SC₁₋₃alkyl, —CO₂H, —CO₂C₁₋₃alkyl, —CONH₂ and —CONHC₁₋₃alkyl;
R₁₀ is selected from —H, —C₁₋₆alkyl, —C₂₋₆alkenyl, —C₂₋₆alkynyl, —C(O)C₁₋₂₀alkyl, —C(O)C₁₋₂₀alkenyl, —C(O)C₁₋₂₀alkynyl, —C(O)C₁₋₆alkylR₁₄, an α-peptide having 1 to 10 α-amino acid residues and an imaging agent;
R₁₀ₐ is selected from —H, —C₁₋₆alkyl, —C₂₋₆alkenyl, —C₂₋₆alkynyl, —C(O)C₁₋₆alkyl, —C(O)C₂₋₆alkenyl, and —C(O)C₂₋₆alkynyl;
each R₁₁ is independently selected from —H, —C₁₋₆alkyl, —C₁₋₆alkylN(R₁₃)₂, —C₁₋₆alkylNR₁₃R₁₄, —C₂₋₆alkenyl, —C₂₋₆alkynyl, —C(O)C₁₋₂₀alkyl, —C(O)C₁₋₂₀alkenyl, —C(O)C₁₋₂₀alkynyl, —C(O)C₁₋₆alkylR₁₄, an α-peptide having 1 to 10 α-amino acid residues and an imaging agent;
each R₁₁ₐ is independently selected from —H, —C₁₋₆alkyl, —C₂₋₆alkenyl, —C₂₋₆alkynyl, —C(O)C₁₋₆alkyl, —C(O)C₂₋₆alkenyl, —C(O)C₂₋₆alkynyl;
R₁₂ is selected from —OH, —OR₁₀ and —N(R₁₁)₂;
each R₁₃ is independently selected from —H and —C₁₋₆alkyl;
R₁₄ is an α-peptide having 1 to 10 α-amino acid residues or an imaging agent;
or a pharmaceutically acceptable salt thereof;
and wherein one of R₁, R₂, R₃, R₄, R₅, R₆ and R₈ comprise a C₈₋₂₀alkyl group, a C₈₋₂₀alkenyl group or a C₈₋₂₀alkynyl group.

The hydrogel comprises one or more β-peptides of formula (I). Accordingly, the hydrogel may comprise one β-peptide (i.e. one species of β-peptide) or a mixture of at least two β-peptides (i.e. two or more species of β-peptide) of formula (I).

In some embodiments, the hydrogel comprises one β-peptide of formula (I). In other embodiments, the hydrogel comprises two β-peptides of formula (I). In yet other embodiments, the hydrogel comprises three β-peptides of formula (I). In yet other embodiments, the hydrogel comprises four β-peptides of formula (I). Advantageously, using more than one species β-peptide may allow the properties of the hydrogel to be modified.

In embodiments where the hydrogel comprises two β-peptides of formula (I), namely a first β-peptide and a second β-peptide, the first β-peptide may be present in a particular amount relative to the second β-peptide by weight. In some embodiments, the hydrogel comprises the first β-peptide and the second β-peptide in a ratio of 3:1, 2:1, 1:1, 1:2, or 1:3 by weight, especially 3:1, 1:1 or 1:3, more especially 1:1. In some embodiments, the first β-peptide is present in an amount of 99%, 98%, 95%, 90%, 85%, 80%, 75%, 70%, 65%, 60%, 55%, 50%, 45%, 40%, 35%, 30%, 25%, 20%, 15%, 10%, 5%, 2%, or 1% by weight, based on the total amount of β-peptide of formula (I). In some embodiments, the second β-peptide is in an amount of 99%, 98%, 95%, 90%, 85%, 80%, 75%, 70%, 65%, 60%, 55%, 50%, 45%, 40%, 35%, 30%, 25%, 20%, 15%, 10%, 5%, 2%, or 1% by weight, based on the total amount of β-peptide of formula (I).

The one or more β-peptides of formula (I) may comprise one or more β-peptides having an α-peptide having 1 to 10 α-amino acid residues and/or an imaging agent, as described above. In some embodiments, the one or more β-peptides of formula (I) comprise one or more β-peptides having an α-peptide having 1 to 10 α-amino acid residues. In some embodiments, the one or more β-peptides of formula (I) comprise one or more β-peptide having an imaging agent. In some embodiments, the one or more β-peptides of formula (I) comprise two β-peptides having an α-peptide having 1 to 10 α-amino acid residues and/or an imaging agent. In some embodiments, the one or more β-peptides having an α-peptide having 1 to 10 α-amino acid residues and/or an imaging agent are present in an amount up to about 100%, 90%, 80%, 75%, 70%, 60%, 50%, 40%, 30%, 25%, 20%, 10%, 8%, 5%, 3%, 2% or 1% by weight, based on the total amount of β-peptide. Advantageously, using a functionalised β-peptide imparts additional functionality to hydrogel. This may allow targeting of certain cells (for example, when the hydrogel comprises a β-peptide of formula (I) having an α-peptide that is cell adhesion motif) and/or visualisation of the hydrogel in assays or in situ (for example, when the hydrogel comprises a β-peptide of formula (I) having an imaging agent such as a fluorophore). The use of multiple functionalised β-peptides provides a multifunctional hydrogel.

The hydrogel may comprise a therapeutic cargo or payload encapsulated within the hydrogel. Advantageously, this allows the hydrogel to function as an encapsulation and delivery system for therapeutics. The therapeutic cargo may be selected from a small molecule drug, a macromolecular drug and a cell. In some embodiments, the therapeutic cargo is a small molecule drug, especially a hydrophilic small molecule drug. The small molecule drug may be any suitable organic compound having a low molecular weight (less than about 900 daltons) and that can regulate a biological process to treat a particular disease. As shown in the examples, the β-peptide hydrogels of the present invention are capable of releasing small molecule compounds encapsulated within the hydrogel. In some embodiments, the therapeutic cargo is a macromolecular drug. Macromolecular drugs useful in the invention include large molecules (molecular weight more than about 900 daltons) such as proteins, polysaccharides and nucleic acids and that can regulate a biological process to treat a particular disease. Examples of suitable macromolecular drugs include proteins such as brain-derived neurotrophic factor (BDNF), glial cell-derived neurotrophic factor (GDNF), catalase and apelin-13, mRNA, microRNA (miRNA), antibodies, and antigens. In some embodiments, the therapeutic cargo is a cell, especially a stem cell, more especially an amniotic cell such as an amniotic epithelial cell or a stromal cell such as a mesenchymal stem cell. The cells may release cargo such as proteins, RNA and cytokines, which diffuse from the hydrogel. As shown in the examples, the β-peptide hydrogels of the present invention are capable of encapsulating cells, delivering cells by injection in vivo and maintaining cell viability. In contrast, hydrogels prepared using β-tripeptides were not capable of encapsulating cells.

In addition to the β-peptides of formula (I), the hydrogel may further comprise one or more β-tripeptides. Suitable β-tripeptides have structural features including an N-terminal that is acylated and presence of a long chain alkyl group such as a C₈₋₂₀ alkyl group. The β-tripeptides may also contain an α-peptide having 1 to 10 α-amino acid residues and/or an imaging agent, as described for the β-peptides of formula (I) described above. Some examples of suitable β-tripeptides include the following:

| No. | Structure |
|---|---|
| 8 | 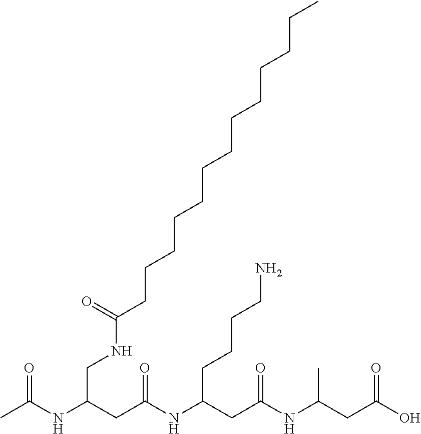 [SMILES: CCCCCCCCCCCCCC(=O)NCC(CC(=O)NC(CCCCN)CC(=O)NC(C)CC(=O)O)NC(C)=O] |
| 9 | 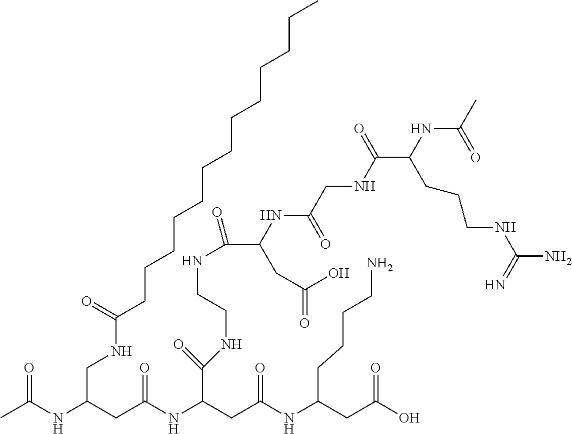 [SMILES: CCCCCCCCCCCCCC(=O)NCC(CC(=O)NC(CC(=O)NC(CCCCN)CC(=O)O)C(=O)NCCNC(=O)C(CC(=O)O)NC(=O)CNC(=O)C(CCCNC(=N)N)NC(C)=O)NC(C)=O] |
| 10 | 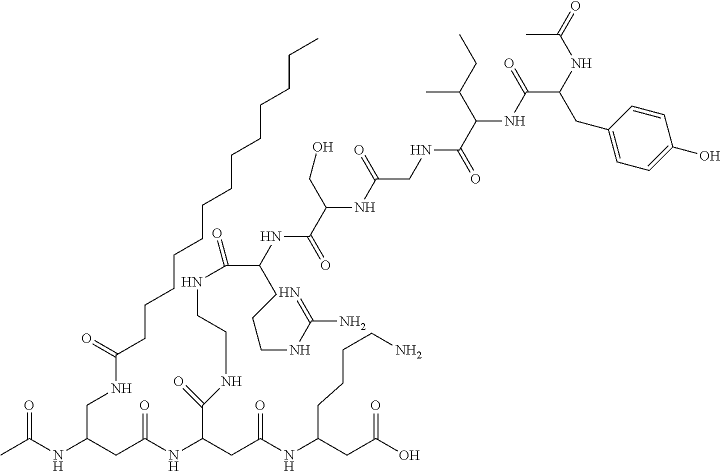 [SMILES: CCCCCCCCCCCCCC(=O)NCC(CC(=O)NC(CC(=O)NC(CCCCN)CC(=O)O)C(=O)NCCNC(=O)C(CCCNC(=N)N)NC(=O)C(CO)NC(=O)CNC(=O)C(NC(=O)C(Cc1ccc(O)cc1)NC(C)=O)C(C)CC)NC(C)=O] |
| | (SEQ ID NO: 6) |
| 11 | 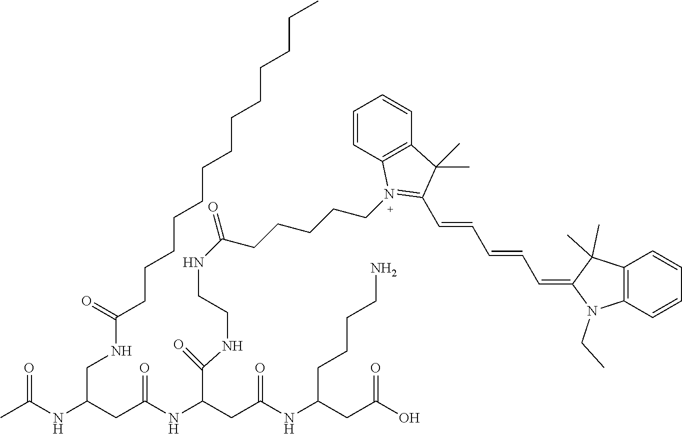 |

Information on the syntheses and uses of β-tripeptides 8 and 9 and 11 can be found in Kulkarni, K., et al., 2016; Kulkarni, K., et al., 2018; and Motamed, S., et al, 2016. β-tripeptide 10 can be prepared following a similar procedure for the synthesis of 9, except that the α-amino acids valine (V), alanine (A), valine (V), lysine (K), isoleucine (I) and serine (S) are coupled to provide the α-peptide.

In some embodiments, the one or more β-tripeptides are present in the hydrogel in an amount up to about 50%, 40%, 30%, 25%, 20%, 10%, 8%, 5%, 3%, 2% or 1% by weight, based on the total amount of β-peptide in the hydrogel. Advantageously, using a mixture of one or more β-peptides of formula (I) and one or more β-tripeptides may allow for modification of the properties of the hydrogel due to the shorter length of the β-tripeptide.

The β-peptide hydrogels of the present invention may advantageously have one or more properties that make them suitable for use systems for encapsulating and delivering therapeutic cargo in vivo. In some embodiments, the β-peptide hydrogel has a gel stiffness that is similar to the viscoelastic properties of biological tissue, especially cerebral tissue. For example, the β-peptide hydrogel may have a plateau storage modulus of from about 0.2 kPa to 20 kPa, especially from about 0.5 kPa to about 15 kPa, in an oscillary time sweep test. As shown in the Examples, β-peptide hydrogels according to the present invention exhibit similar viscoelastic properties to cerebral tissue, which may make the hydrogels suitable for use in the brain. In some embodiments, the β-peptide hydrogels exhibit minimal swelling from the initial gelled state, for example a weight increase of less than 120%, 110%, 100%, 90%, 80%, 70%, 60%, 50%, 40%, 35%, 30%, 25%, 20%, 15% or 10%, compared to the initial weight. As shown in the Examples, β-peptide hydrogels according to the present invention exhibit minimal swelling, which may reduce or avoid the amount of pressure exerted by the hydrogel on surrounding tissue following in vivo implantation. In some embodiments, the β-peptide hydrogels are biocompatible. As shown in the Examples, β-peptide hydrogels according to the present invention are compatible with cells and did not appear to be harmful to cerebral tissue when implanted in vivo. In some embodiments, the β-peptide hydrogels are injectable. As shown the in Examples, when injected into a cavity or vessel, β-peptide hydrogels of the present invention are capable of taking the shape of the cavity or vessel occupied by the hydrogel. In addition, β-peptide hydrogels according to the present invention exhibit favourable shear-thinning properties, where the 3D hydrogel structure collapses under shear stress (such as within a syringe during injection) and quickly recovers to reform the hydrogel once the shear force is removed. This property may be attributed to the hydrogen bonding and non-covalent interactions that form the hydrogel network. Advantageously, this allows the β-peptide hydrogels to be delivered as a liquid along with a therapeutic cargo such as a cell, then gelate after injection to encapsulate the therapeutic cargo.

### 3. Preparation of β-Peptide Hydrogels

The present invention provides methods of preparing β-peptide hydrogels. In some embodiments, the method comprises the step of mixing one or more β-peptides of formula (I) described herein in an aqueous solution to form a hydrogel composition. The hydrogel composition gelates to form the hydrogel.

The hydrogel may comprise a therapeutic cargo encapsulated within the hydrogel. Accordingly, in some embodiments, the method comprises the step of mixing one or more β-peptides of formula (I) described herein and a therapeutic cargo described herein in an aqueous solution to form a hydrogel composition. The hydrogel composition gelates to form the hydrogel, where the therapeutic cargo is encapsulated within the hydrogel.

The amount of β-peptide present in the hydrogel will depend on the amount of aqueous solution present in the hydrogel composition. In some embodiments, the hydrogel comprises the one or more β-peptides of formula (I) in an amount of from about 1 mg/mL to about 20 mg/mL, about 1 mg/mL to about 15 mg/mL, 1 mg/mL to about 10 mg/mL, 5 mg/mL to about 15 mg/mL, or about 5 mg/mL to about 10 mg/mL of hydrogel. In some embodiments, the hydrogel comprises the one or more β-peptides of formula (I) in an amount of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL of the hydrogel.

The aqueous solution is a solution in which the solvent comprises water. In some embodiments, the aqueous solution is selected from water, a buffer or salt solution, especially phosphate buffered saline (PBS), a cell culture medium, especially Dulbecco's Modified Eagle's Medium/Nutrient Mixture F-12 Ham (DMEM/F12), and mixtures thereof. In some embodiments, the aqueous solution is selected from water, a cell culture medium, and mixtures thereof. Advantageously, the presence of one or more salts in the aqueous solution, for example by using a buffer solution or a cell culture medium, may allow for complete gelation of the hydrogel. In some embodiments, the aqueous solution is water. In some embodiments, the aqueous solution is a buffer solution. In some embodiments, the aqueous solution is a cell culture medium. Advantageously, using a cell culture medium may allow encapsulated cells to survive within the hydrogel.

The presence of a salt in the aqueous solution, for example by using a buffer solution or a cell culture medium, may trigger gelation or influence the rate of gelation of the hydrogel. Accordingly, in some embodiments, the aqueous solution has an ionic strength that initiates the gelation of the hydrogel, for example an ionic strength from about 65 to about 98 mM. Other conditions, such as the concentration of β-peptide in the aqueous solution, the pH of the reaction conditions for mixing the aqueous solution and the reaction time may also influence gelation or rate of gelation of the hydrogel.

The hydrogel may further comprise one or more suitable β-tripeptides as described above. Accordingly, in some embodiments, the method comprises the step of mixing one or more β-peptides of formula (I) and one or more β-tripeptides (and optionally a therapeutic cargo) in an aqueous solution to form a hydrogel composition. The hydrogel composition gelates to form the hydrogel.

The β-peptide hydrogels may be prepared in any shape or geometric form. In some embodiments, β-peptide hydrogel is prepared in a form or shape suitable for a particular application of the hydrogel. In some embodiments, β-peptide hydrogel is prepared in a form or shape suitable for a particular mode of administration of the hydrogel. As shown in the examples, hydrogel compositions injected into a cavity or vessel can gelate to form β-peptide hydrogels having the shape of the cavity or vessel occupied by the hydrogel. In addition, hydrogel compositions injected into a liquid can gelate to form β-peptide hydrogels in the form of a hydrogel string in the liquid.

### 4. Applications of β-Peptide Hydrogels

The β-peptide hydrogels of the present invention can be used to deliver a therapeutic cargo. The therapeutic cargo is encapsulated within the hydrogel as described above.

The therapeutic cargo may be released from the β-peptide hydrogel to treat a human or animal disease. The disease to be treated depends on the therapeutic cargo encapsulated in the hydrogel. Accordingly, the therapeutic cargo may be suitably selected for treating a particular disease.

The way in which the encapsulated therapeutic cargo diffuses from the hydrogel may differ depending on the type of therapeutic cargo. Due to their size, encapsulated cells are generally entrapped within the hydrogel but release cargo such as proteins, RNA and cytokines as the hydrogel degrades. For encapsulated macromolecular drugs, a factor that influences diffusion is the mesh size of the hydrogel. The hydrogels are formed of a self-assembled network of self-assembled β-peptide fibers with open spaces (“meshes”) between the fibres. Macromolecular drugs encapsulated within the hydrogel may diffuse from the hydrogel, depending on the size of the drug and the mesh size. The mesh size may be influenced by on the concentration of β-peptide in the hydrogel, as well as external conditions such as temperature, pH and ionic strength.

For encapsulated small molecule drugs, molecular interactions between the β-peptides forming the hydrogel network and the small molecule drugs can influence the release of the drugs from the hydrogel. For example, release can be slowed down, reduced or prevented by forming moderate to strong interactions between the β-peptides and the drugs. Suitable interactions can include electrostatic interactions, for example where the small molecule has a net positive or negative charge and the hydrogel contains one or more β-peptides having an opposing overall net charge at a certain pH. In the case of electrostatic interactions, the β-peptides making up the hydrogel may be suitably selected to have an overall net charge that influences the rate of release of the drug from the hydrogel at a certain pH.

The β-peptide hydrogels of the present invention may be useful in the delivery of cells to treat a human or animal disease. The delivery of stem cells may be particularly useful in the treatment of damaged tissues, such as heart, kidney, liver and brain tissue. As shown in the examples, β-peptide hydrogels of the present invention exhibit similar stiffness to brain tissue, and are capable of maintaining the viability of encapsulated stem cells and delivering the cells to a cerebral infarction in the brain in vivo. Therefore, the β-peptide hydrogels may be useful in the delivery of stem cells to treat stroke.

Accordingly, the present invention provides a method for treating stroke comprising administering to a patient in need thereof a hydrogel comprising one or more β-peptides of formula (I), and a stem cell encapsulated within the hydrogel. The present invention also provides the use of a hydrogel comprising one or more β-peptides of formula (I) and a stem cell encapsulated within the hydrogel in the manufacture of a medicament for treating stroke. The present invention further provides the use of a hydrogel comprising one or more β-peptides of formula (I) and a stem cell encapsulated within the hydrogel in the manufacture of a medicament for treating stroke.

The β-peptide hydrogels of the present invention may be administered by placing the hydrogel directly into the body, for example by surgical implantation or by injection. Advantageously, β-peptide hydrogels according to the present invention may have shear-thinning properties that enable them to be pre-gelled outside the body, and then injected by application of shear stress. This property allows the hydrogels to flow like low-viscosity fluids under shear stress during injection, but quickly recover their initial stiffness after removal of shear stress in the body. In addition, as shown in the examples, β-peptide hydrogels of the present invention are capable of protecting encapsulated cells during injection and can maintain cell viability post-injection. Accordingly, in preferred embodiments, the hydrogel is administered by injection, for example by subcutaneous or intracerebral injection.

### EXAMPLES

### Example 1: Synthesis

### Synthesis of 1 and 2

Ac-Az(Myr)AKS*-Lac (1) and Ac-Az(Myr)AX(RGD)S*-Lac (2) were synthesised on a 0.3 mmol scale using standard Fmoc chemistry on Wang resin (1 mmol/g loading). The resin was swollen in DMF (3 mL) and then soaked in Fmoc-protected β-homo-serine(O^{t}Bu)-OH (2.1 eq. to resin loading), dissolved in DMF (3 mL) along with HATU (2 eq. to resin loading), DMAP (10 mol %), and DIPEA (3 eq. to resin loading), overnight with gentle agitation. The resin was thoroughly washed with DMF (3×3 mL) and the Fmoc protecting group on the amino acid was removed by soaking the resin twice in 20% piperidine in DMF (3 mL) for 15 minutes each. The resin was washed with DMF (3×3 mL), soaked in Fmoc-protected β-homo-lysine(Boc)-OH (2.1 eq. to resin loading) for 1, or (R)-N-Fmoc α-aspartic acid (allyloxycarbonyl)-aminoethyl amide (2.1 eq. to resin loading) for 2, dissolved in DMF (3 mL) along with HATU (2 eq. to resin loading), and DIPEA (3 eq. to resin loading), for 2 hours. β-peptide elongation cycle was then repeated to add β-homo-alanine and β-homo-azidoalanine to the sequence. After removing the terminal Fmoc protecting group on the peptide, the resin was treated with a solution of 10% v/v acetic anhydride and 2.5% v/v DIPEA in DMF (3 mL) for 30 minutes to afford an acetyl-capped N-terminus. The resin was washed with DMF (2×3 mL), CH₂Cl₂ (2×3 mL), Et₂O (2×3 mL), air dried for 10 minutes, and transferred to a 15 mL vial for further manipulation.

For both 1 and 2, to facilitate attachment of the desired aliphatic chain, reduction of the azidoalanine residue was performed on solid support. The resin (0.3 mmol) was swollen in DMF (2 mL) and then soaked in a solution of dithiothreitol (1.23 g, 8 mmol) in DMF (3 mL) and DIPEA (700 μL, 4 mmol), overnight at room temperature with gentle agitation. The resin was washed with DMF (2×3 mL), CH₂Cl₂ (2×3 mL), Et₂O (2×3 mL) and air dried for 10 minutes. The resin was swollen in DMF (2 mL), and the reduction cycle was repeated with a fresh batch of dithiothreitol (1.23 g, 8 mmol) in DMF (3 mL) and DIPEA (700 μL, 4 mmol). The resin was washed with DMF (2×3 mL), then soaked in myristic acid (3.1 eq. to resin loading) dissolved in DMF (4 mL), along with HATU (3 eq. to resin loading) and DIPEA (4.5 eq. to resin loading), for 2 hours. The resin was subsequently washed with DMF (2×3 mL), CH₂Cl₂ (2×3 mL), Et₂O (2×3 mL), air dried for 10 minutes, and transferred to a 15 mL vial for further manipulation.

For 2, to conjugate RGD α-peptide on solid support was preceded by the selective cleavage of the allyloxycarbonyl substituent. CHCl₃ (15 mL) was added to a 50 mL vial and rigorously degassed by bubbling a stream of argon. A portion of the degassed CHCl₃ (˜2 mL) was then used to swell the resin. PhSiH₃ (700 μL) was added to the remaining CHCl₃ (˜8 mL) whilst still bubbling with a stream of argon. Pd(PPh₃)₄ (580 mg, 0.5 mmol) was then added and the mixture was shaken gently until a homogeneous solution was achieved. The resin was then soaked in the Pd(PPh₃)₄ solution for 2 hours, with gentle agitation, and washed with CH₂Cl₂ (3×3 mL) and DMF (3×3 mL) to remove the catalyst. The resin was soaked in Fmoc-protected α-amino acid (3.1 eq. to resin loading), dissolved in DMF (3 mL) along with HATU (3 eq. to resin loading), and DIPEA (4.5 eq. to resin loading), for an hour. The resin was thoroughly washed with DMF (3×3 mL) and the Fmoc protecting group on the amino acid was removed by soaking the resin twice in 20% piperidine in DMF (3 mL) for 15 minutes each. α-Peptide elongation cycle was then repeated until the sequence was complete, with double coupling for Fmoc α-arginine. After removing the terminal Fmoc protecting group on the peptide, the resin was treated with a solution of 10% v/v acetic anhydride and 2.5% v/v DIPEA in DMF (3 mL) for 30 minutes to afford an acetyl-capped N-terminus. The resin was washed with DMF (2×3 mL), CH₂Cl₂ (2×3 mL), Et₂O (2×3 mL), air dried for 10 minutes and transferred to a 15 mL vial for cleavage.

For both 1 and 2, cleavage was performed on resin (0.3 mmol), by treating the resin with a cleavage solution (10 mL) comprising of H₂O (2.5% v/v), triisopropylsilane (2.5% v/v) and 3,6-dioxa-1,8-octanedithiol (0.5% v/v) in CF₃COOH, for 2 hours. CF₃COOH was then evaporated under a stream of N₂, and the peptide was precipitated by addition of Et₂O (50 mL). Both 1 and 2 were separately filtered and the precipitate dissolved in 50% aqueous acetonitrile for lyophilisation. For lactonization, the crude lyophilised peptides 1 and 2, were dissolved in CF₃COOH (10 mL). After 30 min, the acid was evaporated by bubbling a stream of N₂ and the neat residue (without addition or dilution with H₂O) lyophilised immediately. Both 1 and 2 were then separately redissolved in 60% aqueous acetonitrile and purified by preparative HPLC (Agilent HP1200) eluted over a 55 min gradient from 25 to 80% solvent B, (solvent A: 0.1% TFA/H₂O; solvent B: 0.1% TFA/CH₃CN) with a flow rate of 6 mL/min. Fractions were analysed by analytical HPLC (Agilent HP1100), eluted over a 45 min gradient from 0 to 75% solvent B, (solvent A: 0.1% TFA/H₂O; solvent B: 0.1% TFA/CH₃CN) with a flow rate of 1 mL/min, for purity. Molecular mass was confirmed using an Agilent 1100 MSD SL ion trap mass spectrometer. Characterisation data for 1: MS 681.1 (M+H);. Characterisation data for 2: MS 1066 (M+H), 533.6 (M+2H).

### Synthesis of 3 to 7

Ac-Az(Myr)AX(YIGSR)S*-Lac (3) was prepared following a similar procedure for the synthesis of 2 above, except that the α-amino acids arginine (R), serine (S), glycine (G), isoleucine (I) and tyrosine (Y) were coupled to provide the α-peptide and the N terminus of the α-peptide was not acetylated. Characterisation data for 3: 636.7 (M+2H), 424.8 (M+3H);.

Ac-Az(Myr)ASK-OH (4) was prepared following a similar procedure to the synthesis of 1 above, except that Fmoc-β-homolysine(Boc)-OH and Fmoc-β-homoserine(O^{t}Bu)-OH were used in place of Fmoc-β-homoserine(O^{t}Bu)-OH and Fmoc-β-homolysine(Boc)-OH, respectively. Characterisation data for 4: MS 699.3 (M+H).

Ac-Az(Myr)AX(RGD)K—OH (5) was prepared following a similar procedure for the synthesis of 2 above, except that Fmoc-β-homolysine(Boc)-OH was used in place of Fmoc-β-homoserine(O^{t}Bu)-OH. Characterisation data for 5: MS 563.2 (M+2H).

Ac-Az(Myr)AX(YIGSR)K—OH (6) was prepared following a similar procedure for the synthesis of 2 above, except, that the α-amino acids arginine (R), serine (S), glycine (G), isoleucine (I) and tyrosine (Y) were coupled to provide the α-peptide and that Fmoc-β-homolysine(Boc)-OH was used in place of Fmoc-β-homoserine(O^{t}Bu)-OH. Characterisation data for 6: MS 1374 [M+H]688 (M+2H) and 458.9 [M+3H].

Ac-Az(Myr)SX(Quasar)K—OH (7) was also prepared following a similar procedure for the synthesis of 2 above, except that Fmoc-β-homoserine(O^{t}Bu)-OH was used in place of Fmoc-β-homoalanine-OH, Fmoc-β-homolysine(Boc)-OH was used in place of Fmoc-β-homoserine(O^{t}Bu)-OH and Quasar® 570 carboxylic acid (indo-3-carbocyanine N-ethyl-N′-hexanoic acid) was coupled to provide the fluorophore. Characterisation data for 7: MS 612.4 (M+2H).

### Example 2: Atomic Force Microscopy

To confirm the self-assembly of β-tetrapeptides 1 and 2 into fibres, atomic force microscopy (AFM) was performed on a Nanoscope IV AFM with a Multimode head using a vertical engage ‘E’ scanner. A 2 μL peptide solution in MilliQ (ultrapure) water (0.25 mg/mL) was prepared and placed on a clean mica surface. A low concentration of peptide was necessary to avoid contamination the probe. The peptide solution was air-dried for 30 minutes, followed by further drying with a stream of N₂ gas. Images were obtained under tapping mode with NSC-15 ‘B’ silicon cantilevers (Micromasch, Tallinn, Estonia) with a nominal force constant of 40 and 20 N/m for 1 and 2, respectively. Topographic, phase and amplitude images at a resolution of 512×512 were simultaneously obtained using scan frequency of 1 Hz with typical scan sizes of 5 μm×5 μm and 2 μm×2 μm. Images were processed with a sequence of plane fitting and offset flattening using Gwyddion 2.29 (www.gwyddion.net) software.

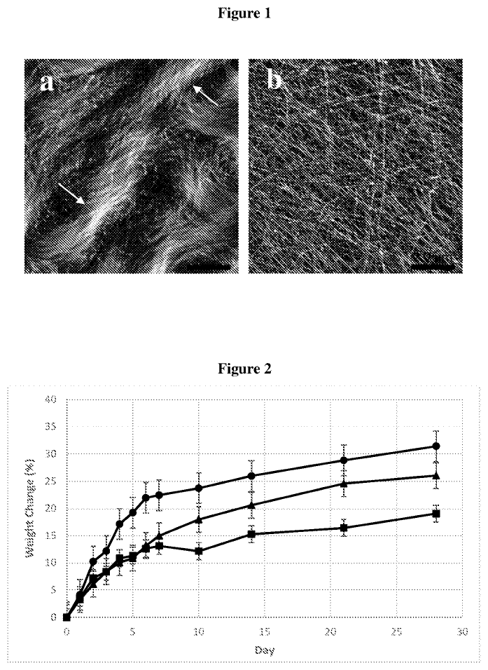

FIG. 1 shows atomic force microscopy images of 1 (FIG. 1a) and 2 (FIG. 1b) dissolved in ultrapure water at 0.25 mg/mL. The spontaneous self-assembly of 1 and 2 into distinct nanofibres was observed, consistent with the behaviour of the previously reported β-tripeptides (Motamed, S., et al., 2016; Kulkarni, K., et al., 2016). The entanglements of the fibres are observably different between 1 and 2. For 1, there was a propensity for the nanofibres to aggregate together and form bundles (FIG. 1a, white arrows), while for 2, the fibres are more wide-spread and mesh-like. This suggests that the presence of the RGD moiety may have an effect the dissolution properties of 2, allowing more even distribution of the fibres within the ultrapure water solvent compared to 1. In addition, 2 required imaging at a lower nominal force compared to 1 as the probe was splitting the nanofibres apart, suggesting that the fibres of 2 were comparatively softer.

### Example 3: Hydrogel Compositions

An inversion test was used to qualitatively assess different solvent conditions and concentrations of 1 and 2 for forming a non-flowing, self-supporting stable hydrogel (Liebman, T., et al., 2007). Peptide solubility was tested using a variety of solvents and solvent mixtures: MilliQ (ultrapure) water, phosphate buffered saline (PBS) 1× (Sigma P3813), PBS 10× (Sigma P3813) and Dulbecco's Modified Eagle's Medium/Nutrient Mixture F-12 Ham (DMEM/F12) (Sigma D8437). 100 μL or 50 μL of solvent was added to 0.5 mg of peptide in a glass vial to achieve a final concentration of 5 or 10 mg/mL, respectively. The solution was vortexed up to 30 mins until all the peptide particles had dissolved and left to self-assemble and gelate for 30 minutes at room temperature. After 30 minutes, the vial was inverted and placed in a 37° C. for 24 hours, at which the gelation result was categorised as follows: liquid (has the same flow as water); viscous liquid (has a more viscous flow compared to water); poor dissolution (the peptide particles did not dissolve completely); unstable gel (initial formation of self-supporting gel following inversion, but the gel was unable to keep its structure after 24 hours); stable gel (initial formation of self-supporting gel following inversion and after 24 hours).

The results from the inversion test for 1 and 2 are summarised in Table 2 below.

TABLE 2

| | Concentration | Gelation result | |
|---|---|---|---|
| Solvent | (mg/mL) | 1 | 2 |
| MilliQ water | 5 | Liquid | Liquid |
| | 10 | Viscous liquid | Liquid |
| PBS 1× | 5 | Viscous liquid | Liquid |
| | 10 | Poor dissolution | Viscous liquid |
| PBS 10× | 5 | Viscous liquid | Liquid |
| | 10 | Poor dissolution | Poor dissolution |
| DMEM/F12 | 5 | Liquid | Liquid |
| | 10 | Poor dissolution | Unstable gel |
| 50% MilliQ | 5 | Unstable gel | Liquid |
| Water/50% | 10 | Unstable gel | Unstable gel |
| DMEM/F12 | | | |
| 75% MilliQ | 5 | Unstable gel | Unstable gel |
| Water/25% | 10 | Stable gel | Stable gel |
| DMEM/F12 | | | |

Regarding the inversion test for 1, 1 readily dissolved in MilliQ water at both 5 and 10 mg/mL concentrations. The higher concentration resulted in a more viscous solution, which could suggest some self-assembly has occurred but not enough for hydrogel formation. 1 was also able to be dissolved in PBS 1× and PBS 10× at 5 mg/mL to provide a viscous liquid but no hydrogel was observed. Increasing the concentration of 1 to 10 mg/mL prevented the peptide from being completely dissolved in either PBS solvent. Gelation began to occur through the combination of MilliQ Water and DMEM/F12. At a 1:1 ratio of MilliQ Water and DMEM/F12, a hydrogel was formed at both 5 and 10 mg/mL concentrations of 1 but the hydrogel would revert back to a solution within 24 hours. Altering the ratio to 3:1 MilliQ Water:DMEM/F12 at 10 mg/mL of 1 resulted in a long-lasting stable hydrogel under physiological conditions over several weeks.

Regarding the inversion test for 2, at 5 mg/mL concentration 2 readily dissolved in MilliQ water, PBS 1×, PBS 10×, DMEM and 50% MQ Water/50% DMEM/F12, but only formed a liquid solution. Increasing the concentration of 2 to 10 mg/mL provided a viscous liquid for PBS 1× but poor dissolution with PBS 10×. Similar to 1, a stable gel was formed with 3:1 MilliQ Water:DMEM/F12 at 10 mg/mL of 2.

The results indicate that suitable hydrogels can be prepared using 1 and 2 in water-based solvent systems. The results also provide an indication that a salt trigger may be useful to initiate complete gelation into a hydrogel. For example, following peptide dissolution in water, the addition of the DMEM/F12 raises the ionic strength of the solution, which may screen the electrostatic interactions between the charged amino acids in the peptide backbone and promote the rapid self-assembly and nanofibre entanglement required for a hydrogel. This system of gelation has been observed in other self-assembling peptide hydrogels including beta-hairpin peptides, diphenylalanine peptides and beta-sheet forming peptides (Mishra, A., et al., 2013; Martin, A. D., et al., 2017; Holmes, T. C., et al., 2000).

### Example 4: Rheological Characterisation

Rheological measurements were undertaken using an Anton Paar Physical parallel plate rheometer at 37° C. Hydrogels were prepared in 75% MilliQ Water/25% DMEM/F12 using 10 mg/mL of the following: 100% 1, 50% 1/50% 2, and 100% 2. The hydrogel solutions were made just prior to experiment and vortexed thoroughly, and then immediately pipetted onto the base. A top plate with diameter 8 mm was lowered to form a measurement gap size of 0.1 mm. In an oscillatory time sweep test, the storage (elastic, G′) and loss (viscous, G″) moduli were measured with strain applied at 1% at a constant frequency of 1 Hz. For the cyclic step-strain test, after 20 minutes, the strain was increased to 100% for 1 minute to mechanically shear the hydrogel before it is returned to 1% strain. This was repeated an additional 2 times. The plateau modulus was calculated as the average of the storage modulus at the end of each 20-minute gelation cycle. A strain sweep was conducted from 1% to 100% strain over the 20 minutes with a constant frequency of 1 Hz. The gel-to-sol transition point was determined at the strain at which the storage and loss modulus equalled in value. A dynamic frequency sweep was performed from 0.01 to 10 Hz with a constant strain of 1%. All measurements were obtained in triplicate and all figures are presented as the average of those triplicates.

The rheological properties of the 3 hydrogels are summarised in Table 3 below.

TABLE 3

| | Plateau Storage Modulus | Strain for gel-to-sol |
|---|---|---|
| Hydrogel | (kPa)^{a} | Transition |
| 100% 1 | 10.2 ± 1.9 | 87% |
| 100% 2 | 5.7 ± 1.9 | 40% |
| 50% 1/50% 2 | 0.8 ± 0.08 | 31% |

^{a}Values = mean ± standard deviation.

The mechanical properties of the prepared hydrogels were characterised via rheological testing. The results of the testing showed that transition from a solution into a viscoelastic material was immediate for all hydrogels, as indicated by the storage modulus being approximately 5 times greater than the loss modulus. The exact point of sol-to-gel phase transition was unable to be pinpointed due to the rapid gelation. After 20 minutes, a plateau was reached at a storage modulus of approximately 10.2 kPa, 5.7 kPa and 0.8 kPa for the hydrogels prepared using 100% 1, 50% 1/50% 2, and 10% 2, respectively, suggesting that increasing the amount of 1 relative to 2 produces a stiffer gel. The difference in stiffness could be attributed to the presence of the RGD moiety of 2, which may create a less dense nanofibre network as it is able to encapsulate and take up more water. This result is in contrast with the rheological behaviour of β-tripeptide hydrogels, where the addition of the RGD moiety caused a 10-fold increase in the storage from 1 kPa to 10 kPa (Kulkarni, K., et al., 2016; and Motamed, S., et al, 2016). The observed values of stiffness are within that of the mean shear modulus of human cerebral tissue in vivo, which has been reported to range from 1.2 kPa to 13.6 kPa depending on the location and pathology (Kruse, S. A., et al., 2008; Sack, I., et al., 2008).

To ascertain the injectability of the hydrogels, a shear thinning test was conducted by subjecting the samples to cycles of strain every 20 minutes. The application of 100% strain disrupted the structure of all hydrogels, causing the reduction of the storage modulus to below that of the loss modulus which indicates gel-to-sol transition. Following cessation of the 100% strain after 1 minute and return to 1% strain, the storage moduli recovered to be higher than that of the loss modulus, thus reverting back to the gel state. The hydrogels were then able to recover over the 20-minute time period to the original storage modulus. This behaviour was highly reproducible over the course of 3 cycles over 84 minutes.

In order to further characterise the shear thinning behaviour of the hydrogels during high strain, a strain sweep was performed from 1% to 100% strain over the course of 20 minutes. The gel-to-sol transition strain was identified as the crossover point where the storage modulus becomes lower than the loss modulus. In agreement with the storage moduli values achieved for each hydrogel, the hydrogel prepared using 100% 1 required the highest strain of 87%, compared to the hydrogel prepared using 50% 1/50% 2 and 2 which required 40% and 31%, respectively.

In order to characterise the stability of the storage modulus over a range of frequencies, a dynamic frequency sweep was performed. The dynamic frequency sweep curves showed the hydrogels' maintenance of their respective linear viscoelastic properties across a range of frequencies. Each hydrogel displayed good stability over the range of tested frequencies, with the storage moduli maintaining at the previously observed plateau values.

The above results indicate that hydrogels prepared using 1 and 2 alone or in combination may be suitable as brain scaffolds. While the strain required to initiate the gel-to-sol transition increased as the stiffness of the gel increased, the difference in stiffness did not affect the shear thinning behaviour, as all of the hydrogels were able to recover immediately following the cessation of high strain. This indicates that shear thinning and recovery mechanisms may be similar for β-tetrapeptide hydrogels of different stiffness.

### Example 5: Swelling Test

The extent that a hydrogel is able to swell to is important when considering implantation in vivo due to amount of pressure it can exert on the surrounding tissue. The degree of hydrogel swelling from the initial gelated state was investigated in DMEM/F12 cell culture media over 1 week. Hydrogels prepared from 10 mg/mL 100% 1, 50% 1/50% 2 and 100% 2 were made at a volume of 20 μL and initially weighed (Mᵢ). 100 μL of DMEM/F12 was carefully added on top of the hydrogels to submerge them, and then the samples were incubated at 37° C. At every 24 hours, the media was carefully removed and drained and the hydrogel was allowed to air dry in the fume hood. Then the swollen mass was weighed (Ms) before the media was replenished. The degree of swelling was calculated by (Mₛ−Mᵢ)/Mᵢ×100. Measurements were obtained in triplicate.

FIG. 2 shows the percentage weight change of the hydrogel prepared using 100% 1 (squares), 50% 1/50% 2 (triangles) and 100% 2 (circles) over time. Minimal swelling of the 3 hydrogels was observed over 28 days, with a 19.04±0.75%, 26.03±1.8% and 31.4±2% weight increase from the initial for the hydrogels prepared using 100% 1, 50% 1/50% 2 and 100% 2, respectively. The percentage weight change of the hydrogels increased as the concentration of 2 increased. This could be due to the increased hydrophilicity of the RGD moiety, which may result in greater encapsulation of water into the peptide network.

The results provide an indication that hydrogels prepared using 1 and 2 may be suitable for use in the brain. The swelling displayed by the hydrogels is low compared to other hydrogels such as those prepared using hyaluronic acid, which can exhibit percentage weight changes ranging from 150% to 600% (Nimmo, C. M., et al., 2011; Collins, M. N. and C. Birkinshaw, 2008). The above results suggest that the β-peptide hydrogels may exhibit minimal swelling inside the stroke infarct of the brain.

### Example 6: In Vitro Injection Test

An in vitro injection model was used to investigate the potential use of the hydrogels as an injectable system. A 0.6 w/v % agarose gel was chosen for the model as it has been shown to be a good in vitro model of the mammalian brain (Pomfret, R., et al., 2013). A solution of 0.6% w/v agarose (Invitrogen UltraPure™ Agarose) was made in PBS 1× and heated to 65° C. using a hot plate until the solution became clear. 1 mL of the solution was then poured into moulds made from 5 mL syringe barrels that had been cut in half and left to cool at room temperature. Just prior to complete setting of the gel, a 1 mL insulin syringe was used to inject air into the agarose to form cavities. After the agarose samples had gelled completely, they were removed from the mould and incubated at 4° C. for 30 mins. The agarose samples were then equilibrated in PBS 1× at 37° C. overnight. Hydrogel samples were prepared using 10 mg/mL of either 1 or 2. The hydrogels also contained 1% trypan blue (Sigma T8154) as a dye. 5 μL of the hydrogel samples were injected into the cavity of the moulds using a 5 μL 23 G microsyringe (SGE Analytical Science) over the course of 3 minutes. The samples were then kept incubated at 37° C. in PBS and the gel morphology was monitored over time.

The hydrogels were able to be prepared and self-assembled prior to being sheared during withdrawal into the microsyringe and subsequently injected and delivered into the fully hydrated brain tissue mimic in the form of agarose gel at 37° C. The hydrogels, stained with trypan blue for visualisation, were able to reassemble after exiting the syringe and formed the shape of the cavity. This integrity was maintained for over a month after being submerged in PBS 1× (pH 7.4) for more than month. These results indicate that the hydrogels have properties that may make them suitable for injection. The results also suggest that when injected into a cavity of defined shape such as a stroke infarct, the hydrogel may be capable of forming a unified structure that conforms to the defect. The results further provide an indication that the hydrogels may be suitable for use in brain tissue.

### Example 7: Qualitative and Quantitative Assessment 3D Encapsulation of Human Amnion Epithelial Cells and In Vitro Cytoviability

To investigate the biocompatibility of the hydrogels and their suitability for encapsulating human amnion epithelial cells, a short term in vitro 3D encapsulation study was performed. The isolation of human amnion epithelial cells (hAECs) from placentas after elective caesarean section at the Hudson Institute of Medical Research was approved by the Monash Health Human Research Ethics Committee (HREC Ref. 12223B), and written informed consent was obtained from the donor. Briefly, the amniotic membrane was carefully removed from the placenta and washed thoroughly in Hanks' Balanced Salt solution (HBSS) (Sigma 55021C) to remove any blood. The membrane was then cut into approximately 4 cm×4 cm pieces, which were incubated in digestion medium (0.05% Trypsin-EDTA) for 10 minutes at 37° C. This is repeated again for 60 minutes. The supernatant was saved and the trypsin inactivated with foetal calf serum (FCS) (ThermoFisher 10437028). After centrifuging, the supernatant was removed and cells resuspended in 10 mL of DMEM/F12+10% FBS. The suspension was strained through a 70 μm filter and resuspended thoroughly. Trypan blue exclusion was used to determine cell number and viability. Cells were frozen in cryopreservation medium of FBS with 5% DMSO, then placed in liquid nitrogen for long term storage.

Hydrogel compositions were prepared using the following amounts of 1 and 2: 100% 1, 95% 1/5% 2, 90% 1/10% 2, 75% 1/25% 2, 67% 1/33% 2, 50% 1/50% 2, 25% 1/75% 2, and 100% 2. Before preparing the hydrogels, 1 and 2 were subjected to a salt exchange to replace the TFA salt with a chloride salt. This was done by dissolving 1 and 2 separately in 0.1 M HCl at 1 mg/mL, vortexing thoroughly and then lyophilising. This process was repeated an additional time. The lyophilised compounds were then dissolved in 40% acetonitrile/60% H₂O at 1 mg/mL and subsequently lyophilised to remove any residual HCl. This step was further repeated twice. To prepare the hydrogels, the lyophilised 1 and 2 were sterilised under UV light for 20 minutes. They were then dissolved in autoclaved, 0.22 μm filtered MilliQ water at a concentration of 12.5 mg/mL and added to wells of a 96-well plate, which was further sterilised under UV light for another 20 minutes. hAECs suspended in DMEM/F12 with 10% FBS and 1% penicillin/streptomycin were added to the hydrogel solutions at a total of 10,000 cells per well. The hydrogel solutions were mixed gently with a pipette to ensure uniform distribution of cells. After incubation at 37° C./5% CO₂ for 30 minutes to stabilise the hydrogels, 100 μL of cell culture media was carefully added on top before incubating further. The media was changed once daily. After 3 days, the hydrogels were rinsed with PBS. A Live Dead assay (Life Technologies) was performed to quantify cell viability by fluorescent microscopy. The live cells were stained with calcein AM and the dead cells with ethidium homodimer according to manufacturer's instructions for qualitative analysis. Images were captured using Nikon Eclipse Ti-U fluorescent microscope. All experiments were done in triplicates.

The fluorescence microscopy images of the Live Dead assay showed that the hAECs were successfully cultured within the hydrogel samples. The majority of non-encapsulated cells were washed away during the media changes and PBS wash, as hAECs tend to be loosely adherent in the initial culture stage. Qualitatively, the cell viability within the different hybrid hydrogels appeared at least on par with the 100% 1 hydrogel, with 50% 1/50% 2 hydrogel having the highest viability. A lower number of viable cells and more noticeable dead cells was observed for the 25% 1/75% 2 and 100% 2 hydrogels.

For a quantitative assessment of cell viability, a (3-(4,5-dimethylthiazol-2-yl)-5-(3-carboxymethoxyphenyl)-2-(4-sulfophenyl)-2H-tetrazolium) (MTS) cell proliferation assay kit was used to measure the cellular metabolic activity after 3 days via reduction of MTS tetrazolium into a coloured formazan product. The absorbance of each well was detected using a plate reader at 490 nm. The cell viability was calculated in terms of percentage relative to the hydrogel sample prepared from 100% 1 in triplicate.

The results of the MTS assay are illustrated in FIG. 3. The results reflect the initial results of the Live Dead assay above. Increasing the amount of 2 (and therefore the amount of the RGD moiety) in the hydrogel generally increased cell viability compared to the 100% 1 hydrogel. The highest cell viability was observed for the 50% 1/50% 2 hydrogel, with almost a 100% increase in cell viability compared to the 100% 1 hydrogel. The cell viability decreased by more than 50% in the 25% 1/75% 2 and 100% 2 hydrogels, suggesting that there may be a toxicity concentration threshold in relation to the amount of 2 used to prepare the hydrogel. A toxicity threshold for RGD has been observed for other cells, where RGD-containing peptides can enter cells directly and upregulate the enzymatic activity of procaspase-3, a pro-apoptotic protein (Buckley, C. D., et al., 1999). This may be the reason for the higher toxicities observed with the 25% 1/75% 2 and 100% 2 hydrogels, as there may potentially be a higher degree of unassembled peptides residing in the hydrogel. It is possible that the most optimally viable hydrogel composition of 50% 1/50% 2 under the conditions of the above assays possessed enough of the RGD moiety to enhance cellular adhesion but not in excess where programmed cell death is promoted.

The above results provide an indication that the hydrogels are capable of encapsulating cells such as hAECs and are able to culture and such cells. While the cells are initially exposed to a hypotonic environment inside the hydrogel following immediate encapsulation, hAECs are capable of tolerating such conditions inside the amniotic cavity in vivo where water from the amniotic fluid is transported through the amniotic membrane to the underlying foetal blood vessels driven by osmotic gradients (Manuelpillai, U., et al., 2012). Furthermore, there are protocols where the amnion is washed in hypotonic solutions to selectively lyse erythrocytes, leaving the hAECs intact (Alitalo, K., et al., 1980).

### Example 8: In Vitro Injection of Hydrogel-Cell System

The viability of encapsulated hAECs being injected was investigated using the hydrogel system of 10 mg/mL 50% 1/50% 2. The hydrogel-cell system was prepared in the same manner as described in the previous example except that 50,000 cells per well were used. For the non-injected (comparison) gels, 5 μL of the hydrogel-cell system was pipetted into the centre of a well of a 96 well cell culture plate. For the injected gels, 5 μL of the hydrogel-cell system was drawn up into a sterilised microsyringe and injected into the centre of the well over the course of 3 minutes. As controls, 5 μL DMEM/F12 cell suspensions containing 50,000 cells and were deposited into the wells in the same manner as the injected and non-injected samples. The hydrogel-cell system was allowed to gelate completely before addition of cell culture media. After incubation at 37° C./5% CO₂ for 30 minutes to stabilise the hydrogel, 100 μL of cell culture media was carefully added on top before returning to the incubator. The media was changed once daily. After 3 days, the hydrogels were rinsed with PBS and a Live Dead assay (Life Technologies) was performed by staining the live cells with calcein AM and the dead cells with ethidium homodimer according to manufacturer's instructions for qualitative analysis. Images were captured using Nikon Eclipse Ti-U fluorescent microscope. All experiments were done in quadruplicates.

The fluorescence microscopy images of the Live Dead assay showed the injected hydrogel system contains viable hAECs. The shape of the injected hydrogel-cell system was less uniform in comparison to the non-injected gel due to the slow injection speeds required to minimise the shear forces inside the needle, however the majority of the hAECs were still encapsulated within the hydrogel. This is in contrast to the non-injected DMEM/F12and injected DMEM/F12 controls, where the cells were more widespread and dispersed since they were not encapsulated within a hydrogel.

A MTS assay was performed to quantify the differences in cytoviability. The same protocol as the previous example was used. The cell viability was calculated in terms of percentage relatively to non-injected DMEM in quadruplicate.

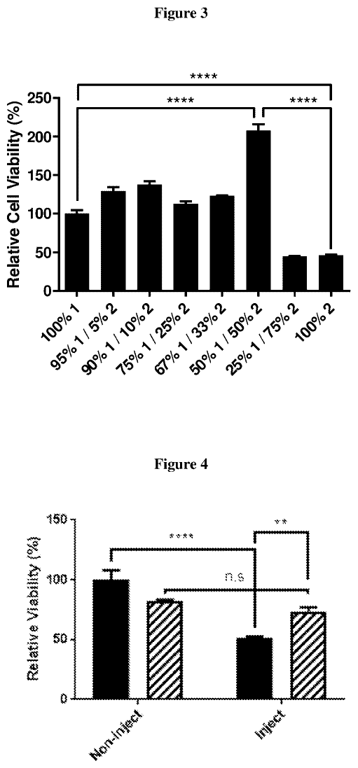

The results of the MTS assay are shown in FIG. 4 (filled bars=DMEM/F12 control, shaded bars=hydrogel-cell system). When the hAECs were injected in only DMEM/F12, there was a 49% decrease in viability compared with the non-injected control. The non-injected hydrogel-cell system saw an approximate 20% reduction in viability compared to the non-injected DMEM control. On the other hand, there was no significant difference between the non-injected and injected hydrogel-cell systems, suggesting that the hydrogel was protective of the hAECs during the injection process.

The above results indicate that the hydrogels may be suitable as an injectable delivery system. The results also provide an indication that the hydrogels are capable of protecting encapsulated cells against shear forces within the syringe needle during injection and can maintain cell viability post-injection.

### Example 9: In Vivo Stroke Studies

The therapeutic potential of hAECs encapsulated within the hydrogel-cell system was investigated in mouse models of photothrombotic stroke after 7 days. The 7 day endpoint study was performed to investigate the acute effects of the various treatment groups on mice models of photothrombotic stroke

### Animals

8 to 10 week old male C57BL/6J mice were obtained from the Monash Animal Research Platform and housed under a 12 hour light-dark cycle with free access to food and water. The animals were divided into five experimental groups: sham, photothrombotic stroke with vehicle treatment (stroke+vehicle), photothrombotic stroke with hydrogel treatment (stroke+gel), photothrombotic stroke with hAECs treatment (stroke+cells), and photothrombotic stroke with hAECs encapsulated hydrogel treatment (stroke+gel+cells). The following groups were assessed: sham (n=6), stroke+vehicle (n=6), stroke+gel (n=8), stroke+cells (n=8), and stroke+gel+cells (n=9).

### Photothrombotic Stroke Model

Under 2-2.5% isoflurane/O₂ gas anaesthesia, mice were placed in a stereotaxic apparatus. The head was shaved and cleaned with ethanol, and a 2 cm-long incision was made in the skin overlaying the cranial midline, exposing the skull. A cold light source attached to give a 2 mm diameter illumination was positioned close to the skull and 1.5 mm laterally right from Bregma. Rose Bengal solution (0.2 mL, 10 g/L in saline) was administered intraperitoneally. After 5 minutes, a small portion of brain was illuminated through the intact skull for 15 minutes, resulting in a stroke spanning the full depth of the cortex, caused by a clot produced when the photosensitive dye in the arteries is excited by the light. This results in impairment of the contralateral (left) forelimb. The skin was then closed using non-toxic glue. Animals are returned to their normal housing conditions. Body temperature was maintained at 37° C. with a heating pad throughout the surgical procedure. In the sham experimental group, the surgery was performed without illumination of the skull, resulting in no infarct.

### Human Amnion Epithelial Cell Culture and Labelling

Primary hAECs were thawed from liquid nitrogen storage and cultured in DMEM/F12 (Sigma) with 10% heat inactivated foetal bovine serum (FBS) and 1% Pen-Strep for 24 hours prior to treatment. Cell count and viability were assessed using trypan blue exclusion, with cells only being used if the viability was at least 90%. Cells were labelled with carboxyfluorescein succinimidyl ester (CFSE) (Life Technologies), a green intracellular dye, to allow for in vivo tracking. Cells were diluted to 5,000 cells/μL for the cell treatment or 6,650 cells/μL for hydrogel encapsulated treatment.

### Hydrogel Preparation

Hydrogels compositions were prepared using 50% 1/50% 2. The hydrogel compositions also contained 2 wt % of a Cy5-tagged β-tripeptide (11) for visualisation. Before preparing the hydrogels, the peptides were subjected to a salt exchange to replace the TFA salt with a chloride salt as per Example 7. The lyophilised peptides were sterilised under UV light for 30 minutes. The hydrogels were prepared by dissolving the peptide powders in 50 vol % MilliQ water before adding 50 vol % of DMEM/F12 to achieve a final concentration of 10 mg/mL. For the hydrogel encapsulated treatment, the DMEM/F12 added was the hAECs cell suspension to achieve a final cell concentration of 5,000 cells/μL for the hydrogel.

### Treatment Injections

Treatment injections were performed at 24 hours post-stroke in order to replicate a real-world scenario, where the patient is at the end of the appropriate time window to receive the conventional stroke treatments of tissue plasminogen activator (tPA) or mechanical clot retrieval. Using a photothrombotic stroke model allow for the creation of a focal ischemia 1.5 mm right of Bregma to predominantly target the M1 cortex, which is responsible for the control and execution of movement such as moving and using forelimbs. 24 hours after stroke induction, the mice were placed in the stereotaxic apparatus again under 2-2.5% isoflurane/02 gas anaesthesia. The skull was re-exposed and a burr hole was drilled 1.5 mm lateral from Bregma. Using a 26-gauge 5 μL MicroVolume syringe (SGE Analytical Science), one of the following was injected directly into the stroke infarct at an approximate rate of 1 μL/min at a depth of 1 mm: 5 μL of saline (for vehicle treatment), 5 μL of hydrogel, 5 μL of 25,000 hAECs or 5 μL of 25,000 hAECs encapsulated within the hydrogel. After injection of the total volume, the needle was kept stationary for an additional 5 minutes and then slowly withdrawn. The skin was then closed with non-toxic glue and the mice were allowed to recover.

### Functional Testing

In order to understand the effect of the treatments on the behavioural deficits and impairments of stroke, functional testing in the form of the hanging wire and cylinder test were performed at days 0 (day of stroke surgery), 1 (day of treatment), 3 and 7. The hanging wire test primarily assesses limb strength, balance and coordination. The test involves suspending the mouse on a wire 60 cm above the ground and measuring the length of time that the mouse is able to hold on, up to a maximum of 300 seconds. This test is performed 3 times per mouse with a 5 minute rest in between. The cylinder test allows for the monitoring of the animals' spontaneous forelimb use to identify forelimb deficits. In the cylinder test, the mouse is placed in a glass cylinder approximately 10 cm in diameter in order to encourage rearing against the cylinder wall using their forelimbs. The mouse is video recorded for 3 minutes, where the use of its forelimbs is counted. A laterality index, which indicates a left or right forelimb preference, was calculated using the following formula:

$$\frac{\begin{matrix}Number of contralateral touches - \\ Number of ipsilateral touches\end{matrix}}{Number of touches with either or both forelimbs}$$

### Histology

The mice were euthanised at day 7. Briefly, the mice were placed in a bell jar containing gauze soaked with isoflurane to render them unconscious. The mice were then decapitated using surgical scissors and the brain was extracted from the skull, fresh frozen in liquid nitrogen and stored at −80° C. The frozen brains were sectioned across the infarct region using a cryostat (Leica) and collected on polylysine coated glass slides to make evenly spaced (˜210 μm apart) 30 μm thick sections for thionin staining to assess infarct size, and 12 μm thick sections for immunohistochemistry. For the latter, the sections were fixed using 4% paraformaldehyde, washed with 1× PBS before permeabilisation with 0.3% Triton X-100. 5% bovine serum albumin (BSA) (Sigma) was used as a blocking solution before the addition of primary antibodies: goat anti-GFAP (1:250, Sigma SAB2500462), rabbit anti-DCX (1:500, Abcam ab18723), and rat anti-F480 (1:500, Bio-Rad Laboratories MCA497G). The sections were incubated overnight at 4° C., then washed with 0.2% Tween-20 (4×5 mins) before the addition of secondary antibodies: Alexa 488-nm-conjugated donkey anti-goat (1:500, Abcam ab150133), Alexa 568-nm-conjugated donkey anti-rabbit (1:500, Abcam ab175692), and Alexa 488-nm-conjugated goat anti-rat (1:500, Life Technologies A11006). Afterwards, sections were washed with 0.2% Tween-20 (3×5 mins), mounted using Vectashield antifade mounting medium (Vector Laboratories) and coverslipped.

### Infarct Volume Calculation

Images of the sections were captured with a CCD camera (Cohu Inc., San Diego, CA, USA) mounted above a light box (Biotec-Fischer Colour Control 5000, Reiskirchin, Germany). Infarct volume was calculated using ImageJ (NIH, Bethesda, MD, USA) by summing the products of the infarct area, the individual section thickness and the distance between each subsequent section to obtain a three-dimensional approximation.

### Fluorescent Image Analysis

Fluorescent images were taken using an Olympus BX51 fluorescent microscope and analysed and quantified also using ImageJ. The number of hAECs within the brain was calculated by summing the product of the number of CFSE fluorescing cells counted within the stroke infarct and peri-infarct area per 30 μm section, the individual section thickness and the distance between each subsequent section to obtain an approximation. For the immunohistochemistry, the total number of target cells or the percentage area based on fluorescence intensity was averaged across three fields of view in the desired region of the tissue section for each animal. Glial scar width was calculated using ImageJ by creating a linear plot profile perpendicular across the glial scar and measuring the length of increased fluorescent intensity. This is performed three times per section and averaged. Then, the mean of the averages of each section for each animal was calculated.

### Statistical Analysis

All data are presented as mean±SEM. Statistical analyses were performed using GraphPad Prism version 8 (GraphPad Software Inc., San Diego, CA, USA). Inter-treatment group comparisons were compared using one-way analysis of variance (ANOVA) (for three or more groups) or Student's unpaired t test (two groups), as appropriate. The * symbol directly above a column or data point in the presented figures refers to significance against the stroke+vehicle control.

### Results—7 Day Post-Stroke Study

### Functional Testing

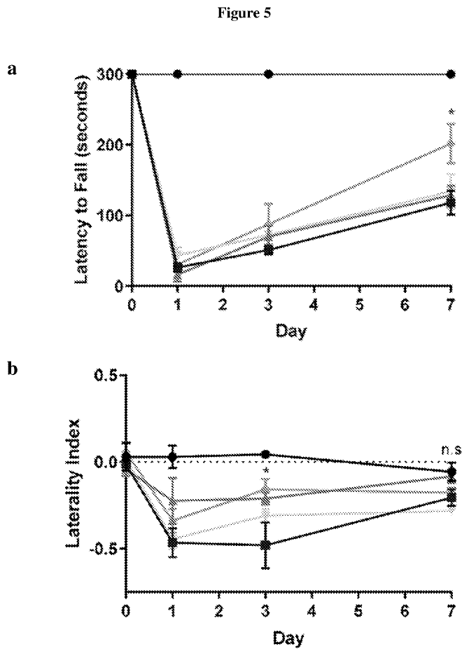

The results of the hanging wire test are shown in FIG. 5a for the following treatments: sham (circles), vehicle (squares), hydrogel (up triangles), hAEC (down triangles), and hydrogel encapsulated hAEC (diamonds). At day 0, prior to stroke surgery, a baseline time for each mouse was obtained at the maximum 300 seconds, which the sham mice maintained throughout the testing period. Post stroke surgery at day 1, the latency to fall decreased dramatically for each mouse to approximately 50 seconds, indicating severe motor impairments. A common observation in each mouse following stroke was constant tremors in the impaired contralateral left forelimb. Through the period to day 7, all the animals had sustained improvements to their latency to fall times. However, significant motor functional recovery was only observed in the mice treated with the hydrogel encapsulated with hAECs at day 7 compared to the other treatment groups, as indicated by the increased hanging time of approximately 80 seconds (p<0.05).

The results of the cylinder test are shown in FIG. 5b for the following treatments: sham (circles), vehicle (squares), hydrogel (up triangles), hAEC (down triangles), and hydrogel encapsulated hAEC (diamonds). The laterality index indicates forelimb preference or handedness. For example, a score of −1 signifies complete preference for the right forelimb, +1 signifies complete preference for the left forelimb, 0.0 signifies no preference (uses both forelimbs equally). At day 0, prior to stroke surgery, a baseline laterality of approximately 0.0 was observed for all mice. At day 1 post-stroke, the laterality index becomes negative for all stroke mice, indicating that they have an increased preference for the right forelimb due to impairment in their left forelimb. At day 3 post-stroke, mice treated with hydrogel encapsulated hAECs had an index of −0.15 compared mice treated with with vehicle that had an index of −0.48 which was a significant improvement in forelimb symmetry. At day 7, all treatment groups had no significant difference between them, converging on an index of approximately −0.1. This suggests that while the animals became less asymmetrically reliant on their right forelimb over the course of 7 days, the mice treated with the hydrogel encapsulated hAECs had a slightly faster recovery.

### Infarct Characterization and Volume

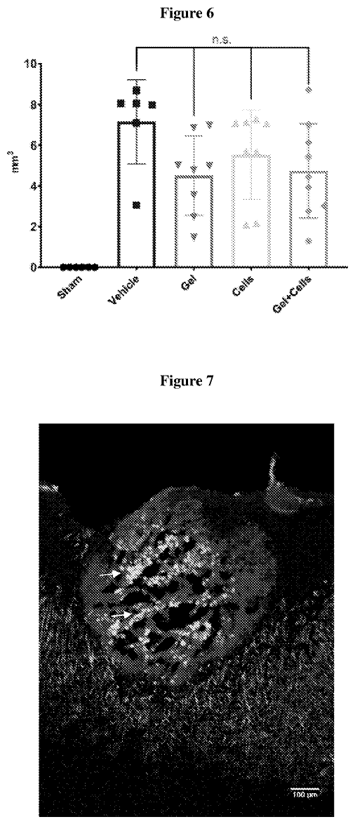

Thionin staining, which stains for acidic proteins and nucleic acids, allows for the visualisation of the whole brain and the infarct for both qualitative and quantitative analysis, including validation of the retention of the injected hydrogel within the infarct cavity. In contrast with the healthy tissue, the stroke cavity is clearly distinct in the brains of the sham, vehicle-treated, hydrogel-treated, hAEC-treated and hydrogel encapsulated hAEC-treated mice at day 7, located within the right cortex of the brain. In the hydrogel encapsulated hAEC-treated mouse, the injected hydrogel formed an ovoid-like shape directly within the cavity. FIG. 6 illustrates quantification of the infarct volume (mm³) for each treatment. These results showed that treatment with hAECs, or hydrogel, or hydrogel encapsulated hAECs resulted in a smaller infarct volume compared to the vehicle, however no statistical difference was found between the treatment groups. This suggests that treatment with the hydrogel encapsulated hAECs, as well as treatments with the hydrogel and hAECs alone, did not have an effect on infarct volume.

Visualisation of the Hydrogel Encapsulated Cells within the Infarct

The hydrogel can be easily identified within the infarct due to the addition of the Cy5-labelled β-tripeptide in the hydrogel composition, allowing visualisation in the far-red wavelength. FIG. 7 is a fluorescence microscopy image of the infarct of the brain of a mouse treated with hydrogel encapsulated hAECs. The fluorescent hydrogel appears red (colur not shown), the hAECs within the hydrogel appear yellow (colour not shown), the DAPI/nucleus appears blue (colour not shown), and the glial fibrillary acidic protein (GFAP) appears green (colour not shown). The hAECs (white arrows) are shown to be clearly within the core of the hydrogel on what appears to be prominent microfibers of self-assembled β-peptide. There is cellular infiltration into the hydrogel from the native tissue, as shown by the blue DAPI staining on the edges of the hydrogel, which are most likely infarct cell debris or immune cell such as microglia and macrophages.

### Cell Count

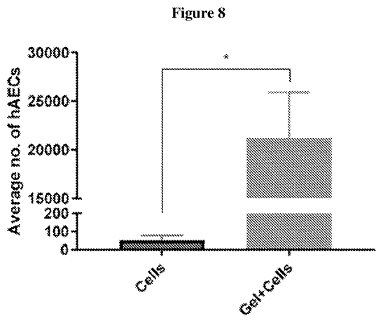

To investigate whether the hydrogel was effective in encapsulating the injected hAECs, the number of detectable fluorescent hAECs that still possessed the CFSE label were visualised and counted. Fluorescence microscopy images of the infarct of the brain of a mouse treated with hAECs and a mouse treated with hydrogel encapsulated hAECs showed the detectable fluorescent hAECs in green. The fluorescence of the hydrogel was omitted to allow for better visualisation of the hAECs. FIG. 8 illustrates quantification of the average number of hAECs counted inside the brain. The number of hAECs able to be detected was significantly increased by a magnitude of 400 (p<0.05) in the brains of the mice treated with hydrogel encapsulated cells compared to those treated with cells alone. This suggests that the hydrogel increased the longevity of the cells within the infarct. No hAECs were able to be detected outside the infarct itself.

### Glial Scar Characterisation

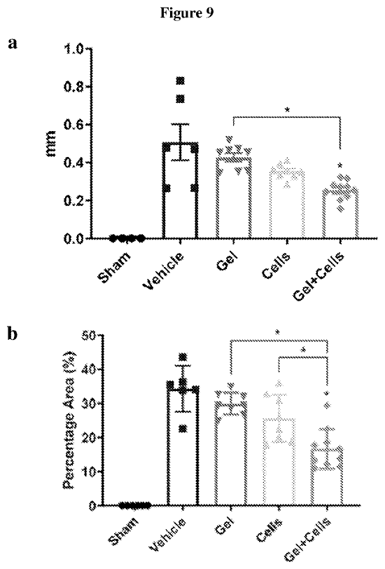

Glial fibrillary acidic protein (GFAP) can be used to stain for astrocytes inside the brain. During stroke, astrocytes become activated and create a glial scar surrounding the infarct. This creates a barrier that walls off the damaged area from the rest of the brain but also hinders neuroregeneration and reinnervation, disrupting recovery. This is evident in the fluorescence microscopy images of GFAP-stained brains of the vehicle-treated, hAEC-treated, hydrogel-treated and hydrogel encapsulated hAEC-treated mice at day 7. FIG. 9a illustrates quantification of the average glial scar width (mm) and FIG. 9b illustrates quantification of the density of activated astrocytes within the glial scar. Mice treated with the hydrogel encapsulated hAECs resulted in a 50% reduction in the average glial scar width and 53% reduction in the density of activated astrocytes within the glial scar (p<0.05) compared to vehicle. Treatment with hAECs alone also had a significant effect, with a decrease of 30% and 25% in the glial scar width and density, respectively, compared to the vehicle (p<0.05). The hydrogel treatment alone showed no significant difference compared to the vehicle (p>0.5).

The above results show that direct delivery of hydrogel encapsulated hAECs extends the longevity and retention of the cells within the stroke infarct. The results also showed that the hydrogel encapsulated hAEC-treated mice exhibited functional improvement in the functional tests, reduction in local glial scarring and increased neuroblast migration and regeneration. These results provide an indication that the hydrogel-cell system may be useful in the treatment of stroke.

### Example 10: Qualitative Assessment 3D Encapsulation of Human Amnion Epithelial Cells and In Vitro Cytoviability

An in vitro 3D encapsulation study was performed to compare the suitability of the hydrogels prepared using β-tetrapeptides or β-tripeptides for encapsulating hAECs and maintaining cell viability after encapsulation. For the β-tetrapeptide hydrogel, the following hybrid hydrogel composition was prepared: 50% 1/50% 2 at 10 mg/mL. For the β-tripeptide hydrogel, the following hybrid hydrogel composition was prepared: 50% 8/50% 9 at 10 mg/mL. As a control, hAECs on tissue culture polystyrene was used.

The hydrogel-cell systems were prepared in the same manner as described in Example 7 except that 30,000 cells per well were used. After 3 days, the hydrogels were rinsed with PBS and a Live Dead assay (Life Technologies) was performed by staining the live cells with calcein AM and the dead cells with ethidium homodimer according to manufacturer's instructions for qualitative analysis. Images were captured using Nikon Eclipse Ti-U fluorescent microscope. All experiments were done in quadruplicates.

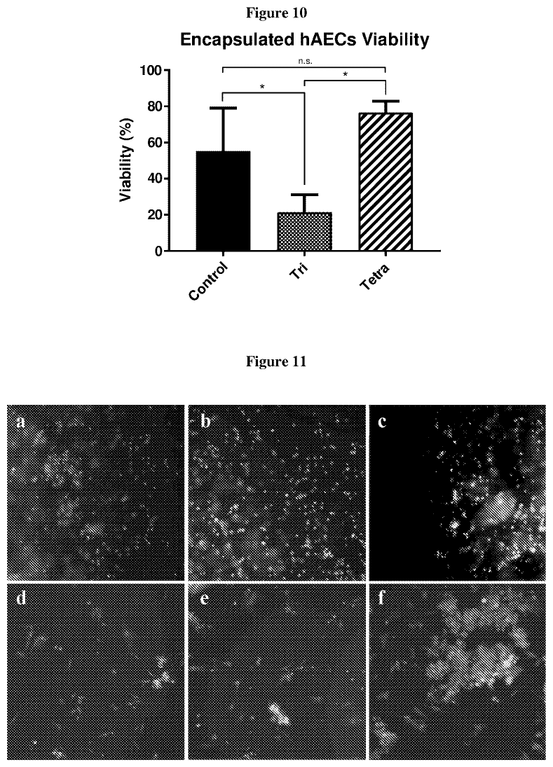

The results of the assay are shown in FIG. 10. The cell viability was calculated in terms of percentage of live cells to dead cells within each treatment in quadruplicate. A significant increase in cell viability was observed for the control and β-tetrapeptide hydrogel when compared to the β-tripeptide hydrogel (p<0.05). There was no significant difference between the viability of the control and β-tetrapeptide hydrogel. These results provide an indication that β-tripeptide hydrogels are not capable of supporting cells within the 3D hydrogel structure, whereas β-tetrapeptide hydrogels can encapsulate and support the viability of cells such as hAECs within the hydrogel.

### Example 11: Qualitative Assessment 3D Encapsulation of Mesenchymal Stem Cell and In Vitro Cytoviability

A short term in vitro 3D encapsulation study was performed to investigate the suitability of the hydrogels for encapsulating mesenchymal stem cells (MSC) and maintaining cell viability after encapsulation. As a comparison, hydrogels prepared using different β-tripeptides in different amounts were also evaluated.

For the β-tetrapeptide hydrogels, the following hybrid hydrogel compositions were prepared: 80% 1/20% 2 and 50% 1/50% 2. For the β-tripeptide hydrogels, the following hybrid hydrogel compositions were prepared:100% 8, 95% 8/5% 9, 90% 8/10% 9, 80% 8/20% 9, 50% 8/50% 9, 100% 9, 95% 8/5% 10, 90% 8/10% 10, 80% 8/20% 10, 50% 8/50% 10 and 100% 10. Before preparing the hydrogels, 1, 2, 8, 9 and 10 were subjected to a salt exchange to replace the TFA salt with a chloride salt. This was done by dissolving 1, 2, 8, 9 and 10 separately in 0.1 M HCl at 1 mg/mL, vortexing thoroughly and then lyophilising. This process was repeated an additional time. The lyophilised compounds were then dissolved in 40% acetonitrile/60% H₂O at 1 mg/mL and subsequently lyophilised to remove any residual HCl. This step was further repeated twice. To prepare the hydrogels, the lyophilised 1, 2, 8, 9 and 10 were dissolved in MilliQ water at a concentration of 60 mM. CFSE-stained MSCs suspended in phenol-free alpha minimum essentials medium (aMEM) were added by gentle pipetting to the hydrogel at a total concentration of 30 mM at 1 million cells/mL density. The hydrogel was then pipetted into a tissue culture plate and allowed to set for 5 minutes incubation at 37° C./5% CO₂ before 200 μL of phenol-free cell-culture media (aMEM, 16.5% FBS, 1% Pen-Strep, 1% glutamine) was added on top. After 24 hours the hydrogels were rinsed with PBS. A modified Live Dead assay (Life Technologies) was performed to indicate cell viability by fluorescent microscopy. The dead cells were stained with ethidium homodimer according to manufacturer's instructions for qualitative analysis. Images were captured using Nikon C1 Invert confocal microscope. All experiments were done in triplicate.

The results of the Live Dead assay are shown in FIG. 11 for the following hydrogel compositions: 80% 8/20% 9 (FIG. 11a), 80% 8/20% 10 (FIG. 11b), 50% 8/50% 10 (FIG. 11c), 80% 1/20% 2 (FIG. 11d), and 50% 1/50% 2 (FIGS. 11e and 11f). Live cells appear green and dead cells appear red (colour not shown). For each of the β-tripeptide hydrogels, almost all MSCs appeared red (colour not shown) indicating that they were dead. On the other hand, the β-tripeptide hydrogels were found to support MSC survival to varying degrees. These results provide an indication that β-tripeptide hydrogels are not capable of supporting cells within the 3D hydrogel structure, whereas β-tetrapeptide hydrogels can encapsulate and support the viability of cells such as MSCs within the hydrogel.

### Example 12: Hydrogel Encapsulated Drug Release Studies

Trypan Blue Release from Bulk Hydrogels

To investigate the suitability of the hydrogels for releasing drugs encapsulated within the hydrogel, a drug release study was performed using trypan blue as a model drug. To prepare the hydrogel compositions, 1 and 2 powders were mixed to obtain 1 mg peptide compositions in the following weight ratios: 100% 1, 75% 1/25% 2, 50% 1/50% 2, 25% 1/75% 2, and 100% 2. The peptide compositions were then separately dissolved in 75 μL of MilliQ water, before adding 25 μL of 1× PBS containing 40 μg or 41.67 nmol of trypan blue dye (Sigma) to trigger the self-assembly and gelation of hydrogel containing 10 mg/mL peptide. 30 μL of the hydrogel solution was pipetted into 3 separate wells of a 48 well plate (Corning) to obtain triplicates, providing 12 μg or 12.5 nmol of trypan blue dye encapsulated within each hydrogel sample. The hydrogels were incubated at 37° C. for 10-15 minutes to allow for complete gelation, before the addition of 300 μL of 1× PBS at pH 7.4 into each well. The hydrogels were kept at 37° C. for the duration of the study. The PBS was collected and replaced with fresh 1× PBS at various time points (after 1, 2, 3, 4, 5, 6, 7, 14 and 21 days) and frozen at −20° C. Once all samples were collected, a Multiskan Spectrum plate reader (Thermo Fisher) was used to determine the amount of trypan blue dye in each sample by measuring the absorbance of the dye at 590 nm.

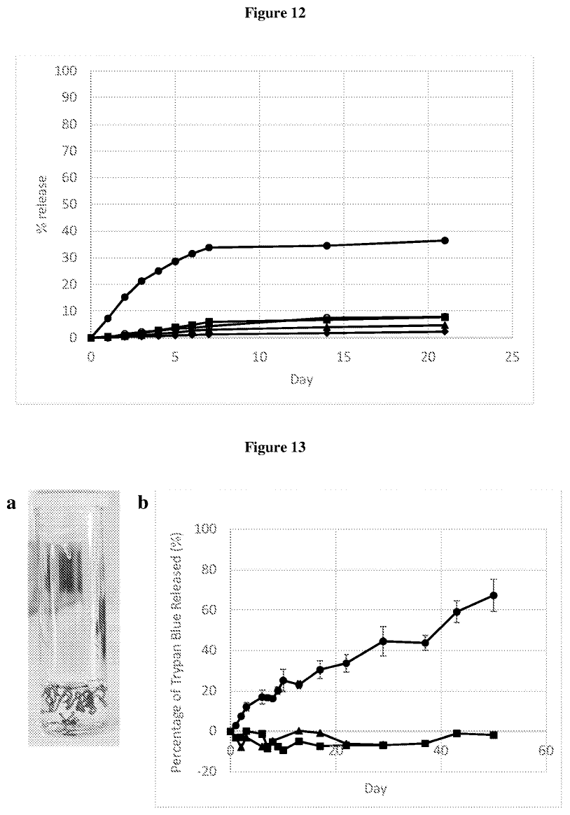

FIG. 12 shows the release of trypan blue from the following hydrogel samples over time: 100% 1 (hollow circles), 75% 1/25% 2 (diamonds), 50% 1/50% 2 (triangles), 25% 1/75% 2 (squares), and 100% 2 (filled circles). Release was only observed with the 100% 2 hydrogel, which reached a plateau after 7 days with approximately 35% release of trypan blue. This result indicates that an increased amount of 1 hinders the release of trypan blue. This effect can be attributed to the overall positive charge of 1 at pH 7.4 due to the presence of the lysine sidechain. Since trypan blue is negatively charged, it would form a charge-charge interaction with 1, leading to the retention of the dye within the hydrogel in the presence of 1. On the other hand, 2 has an overall net neutral charge at pH 7.4 and therefore would not form a charge-charge interaction with trypan blue, resulting in release of trypan blue from the hydrogel in the presence of 2 only. These results provide an indication that the hydrogels may be suitable for use as a drug delivery vehicle and reservoir for bioactive agents. The results also suggest that the β-peptides used to prepare the hydrogel and their respective amounts can influence the release of charged molecules encapsulated in the hydrogel.

Trypan Blue Release from String Hydrogels

To investigate the suitability of the hydrogel in the form of a hydrogel string for releasing encapsulated drugs, a drug release study was performed using trypan blue as a model drug. To prepare the hydrogel compositions, 1 and 2 powders were mixed to obtain 1 mg peptide compositions in the following weight ratios: 100% 1, 50% 1/50% 2, and 100% 2. The peptide compositions were then separately dissolved in 100 μL of MilliQ water containing 40 μg or 41.67 nmol of trypan blue dye (Sigma). 30 μL of the hydrogel solution was pipetted into 3 separate wells containing 1 mL of 1× PBS at pH 7.4 of a 48 well plate (Corning) to obtain the hydrogel strings in triplicates, providing 12 μg or 12.5 nmol of trypan blue dye encapsulated within each hydrogel string. The hydrogels were incubated and kept at 37° C. for the duration of the study. The PBS was collected and replaced with fresh 1× PBS at various time points (after 1, 2, 3, 6, 7, 8, 9, 10, 13, 17, 22, 29, 37, 43 and 50 days) and frozen at −20° C. Once all samples were collected, a Multiskan Spectrum plate reader (Thermo Fisher) was used to determine the amount of trypan blue dye in each sample by measuring the absorbance of the dye at 590 nm.

An image of prepared hydrogel strings with encapsulated trypan blue in 1× PBS is shown in FIG. 13a. FIG. 13b shows the release of trypan blue from the following hydrogel string samples over time: 100% 1 (circles), 50% 1/50% 2 (triangles), and 100% 2 (squares). Similar to the release profiles of the hydrogels in the previous experiment, release was only observed with the 100% 2 hydrogel sample. There appeared to be sustained release of the encapsulated trypan blue from the 100% 2 hydrogel over the 50 day period (dotted line). These results show that the hydrogels may be prepared in alternative forms such as hydrogel strings. The results also show that the encapsulated trypan blue was released from the hydrogel string in a similar manner to the bulk hydrogel in the previous example. This suggests that the hydrogel in the form of a hydrogel string may also be suitable as a drug delivery vehicle or reservoir.

Trypan Blue Release from String Hydrogels Under Acidic and Basic pH Conditions

To investigate the effects of pH on the release of drugs encapsulated in the hydrogels, a drug release study was performed using trypan blue as a model drug. To prepare the hydrogel compositions, 1 and 2 powders were mixed to obtain 1 mg peptide compositions in the following weight ratios: 100% 1, 50% 1/50% 2, and 100% 2. The peptide compositions were then separately dissolved in 100 μL of MilliQ water containing 40 μg or 41.67 nmol of trypan blue dye (Sigma). 30 μL of the hydrogel solution was pipetted into 3 separate wells containing either 1 mL of 1× PBS at pH 5 or pH 9 of a 48 well plate (Corning) to obtain the hydrogel strings in triplicates for each pH, providing 12 μg or 12.5 nmol of trypan blue dye encapsulated within each hydrogel sample. The hydrogels were incubated and kept at 37° C. for the duration of the study. The PBS was collected and replaced with fresh 1× PBS of the corresponding pH at various time points (after 1, 2, 3, 6, 7, 8, 13, 17 and 22 days) and frozen at −20° C. Once all samples were collected, a Multiskan Spectrum plate reader (Thermo Fisher) was used to determine the amount of trypan blue dye in each sample by measuring the absorbance of the dye at 590 nm.

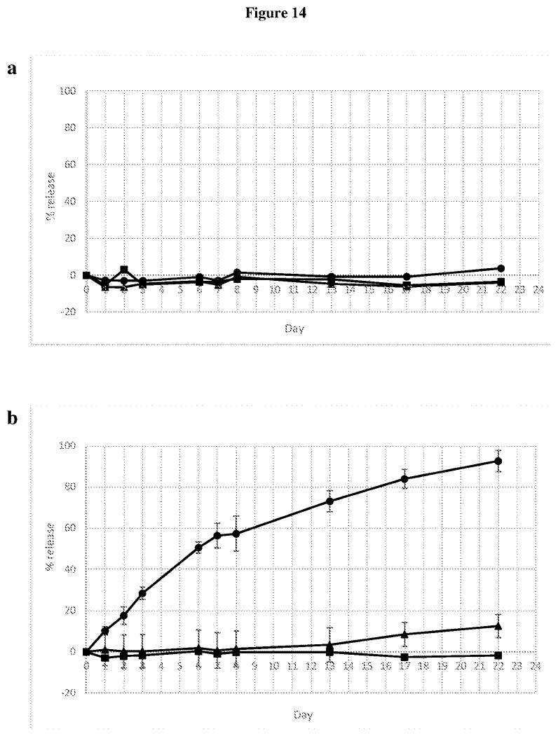

FIG. 14 shows the release of trypan blue at pH 5 (FIG. 14a) or pH 9 (FIG. 14b) from the following hydrogel string samples over time: 100% 1 (squares), 50% 1/50% 2 (triangles), and 100% 2 (circles). At an acidic pH of 5, no release of trypan blue was observed for any of the hydrogel string samples. At a basic pH of 9, release was only observed for the 100% 2 hydrogel sample, where the amount of trypan blue released was higher and the rate of release was faster compared to the corresponding hydrogel sample at pH 7.4. These trends can be attributed to the change in the charge state of 2 under the acidic and basic conditions. At both pH 5 and pH 9, 1 still has an overall positive charge and would still form a charge-charge interaction with the trypan blue. Regarding 2, at pH 5, the arginine sidechain of the RGD moiety becomes positively charged, resulting in 2 having an overall positive charge at this pH. As a result, both 1 and 2 would form charge-charge interactions with trypan blue at pH 5, leading to the retention of the dye within the hydrogels containing 1 or 2 or both. At pH 9, the arginine sidechain of the RGD moiety would become neutral but the aspartic acid sidechain becomes negatively charged, resulting in 2 having an overall negative charge at this pH. This would lead to repulsion between the negatively charged 2 and trypan blue, and therefore increased release and rate of release of encapsulated trypan blue from the hydrogel in the presence of increased amounts of 2. These results provide an indication that the hydrogel strings may be suitable for use as a drug delivery vehicle and reservoir for bioactive agents. The results also suggest that pH conditions can influence the release of charged molecules encapsulated in the hydrogel.

DNA Primer Release from String Hydrogels

To investigate the suitability of the hydrogels for releasing charged payloads encapsulated within the hydrogel, a drug release study was performed using a DNA primer sequence as a model payload. To prepare the hydrogel compositions, 1 and 2 powders were mixed to obtain 1 mg peptide compositions in the following weight ratios: 100% 1, 50% 1/50% 2, and 100% 2. The peptide compositions were then separately dissolved in 100 μL of MilliQ water containing 6.6 μg or 1 nmol of DNA primer of sequence 5′ CAA GCT CAA TGT CCT TCC ACT T 3′. 30 μL of the hydrogel solution was pipetted into 3 separate wells containing 0.3 mL of DMEM at pH 7.4 of a 48 well plate (Corning) to obtain the hydrogel strings in triplicates. The hydrogels were incubated and kept at 37° C. for the duration of the study. The 1× DMEM was collected and replaced with fresh 1× DMEM at various time points and frozen at −20° C. Once all samples were collected, a DeNovix DS-11 FX Spectrophotometer was used to measure the amount of DNA primer in each sample.

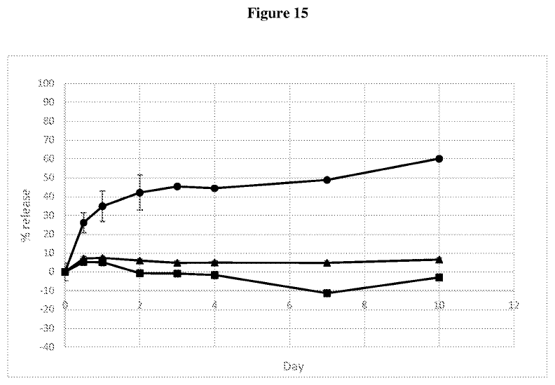

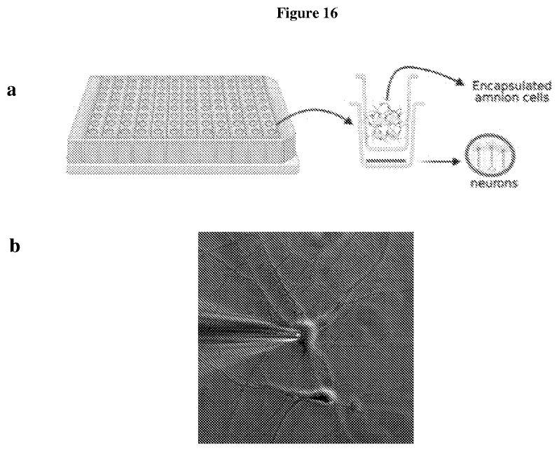

The DNA primer has an overall negative charge due to the negatively charged phosphate backbone of the nucleotides. The results were similar to those obtained for release of encapsulated trypan blue, which also has an overall negative charge, from hydrogel strings at pH 7.4. FIG. 15 shows the release of the DNA primer from the following hydrogel samples over time: 100% 1 (squares), 50% 1/50% 2 (triangles), and 100% 2 (circles). Release of DNA primer was only observed with the 100% 2 hydrogel sample. These results suggest that the hydrogel in the form of a hydrogel string may be suitable as a drug delivery vehicle or reservoir. The results also suggest that the β-peptides used to prepare the hydrogel and their respective amounts can influence the release of charged molecules encapsulated in the hydrogel.

### Example 13: In Vitro Neuron, Hydrogel and hAEC Hydrogel Co-Culture Studies

The modulatory potential of human amnion epithelial cells (hAECs) encapsulated within the hydrogel-cell system was investigated in primary hippocampal neuron co-culture assays. Co-cultures were assayed at the 4 day and 7 day timepoints in order to investigate the changes in neuron function and anatomy over time.

### Hydrogel Preparation

The hydrogel-cell systems were prepared in the same manner as described in Example 7 except that 15,000 or 25,000 hAECs per well were used.

### Primary Hippocampal Neuron Co-Culture

Hippocampal cell cultures: Primary hippocampal neurons from embryonic day 18 (E18) rats were used for in vitro studies. These procedures were approved by the Monash University Animal Ethics Committee and conform to the Australian National Health and Medical Research Council code of practice for the use of animals in research. Pregnant rats (day 18) were deeply anaesthetized using isoflurane, rapidly decapitated using a guillotine and brains were isolated from the pups. Hippocampi were dissected free and single cells were isolated using trypsin (0.25 mg/mL) (Sigma-Aldrich). The cells were resuspended in Dulbecco's Modified Eagle's Medium (DMEM) (Gibco) containing 10% fetal calf serum (FCS) and penicillin/streptomycin (100 U/mL), and seeded (250,000 cells/mL) onto sterilized samples, one per well in a 24-well plate. Two hours later, when cells had adhered to the substrate, Neurobasal A medium (1 ml) containing pen/strep (100 u/mL), glutamine (2.5 mM) and B27 (2%) (all from Invitrogen) was added to each well. Fifty percent of the medium was replaced every 3 days. The harvested cells consisted of a mixed population of neurons and astrocytes, with a high neuron percentage. A mixed neuron and astrocyte population mimics the natural neuronal environment. All cell culture reagents used were purchased from Invitrogen (Mulgrave, Australia) unless specified below.

### Electrophysiology

Electrophysiology of cultured cells: 3D-PEDOT and glass-coverslips containing cells were transferred to a perfusion bath at room temperature (RT), and continuously superfused with physiological saline solution (PSS) containing (mM): NaCl 137, NaHCO₃4, NaH₂PO₄ 0.3, KCl 5.4, KH₂PO₄ 0.44, MgCl₂ 0.5, MgSO₄ 0.4, glucose 5.6, HEPES 10, CaCl₂) 1.5 at pH 7.4. Glass electrodes, 2-5MΩ resistance, were pulled, fire polished and filled with solution containing (mM): KCl 10, CaCl₂ 0.05, Mg₂ATP 4, K-Gluconate 130, Na₂-Phosphocreatine 10, EDTA 0.01, EGTA 0.1, HEPES 10, pH 7.2. Electrophysiological activity was recorded using the patch clamp technique in whole-cell mode using an Axopatch 200A series amplifier controlled by pCLAMP v.10 software. Data were digitized at 5-20 kHz and analyzed using Clampfit 10 (Axon Instruments). Current clamp mode was used to record the resting membrane potential, input capacitance and resistance. Action potentials were evoked using depolarizing current steps. Voltage clamp mode using 10 mV depolarizing steps, from a holding potential of −100 mV, was used to record voltage gated inward currents. Tetrodotoxin (TTX) was used to interrupt Na⁺ currents (Sigma-Aldrich). The preparations were fixed in paraformaldehyde 4% immediately upon termination of electrophysiology and were later stained immunohistochemically.

### Immunohistochemistry

Immunohistochemistry: Neurons cultured for various DIV on glass, 2D-PEDOT and FM and fiber mats were studied using electrophysiology and/or calcium imaging and subsequently fixed in 4% PFA and stored briefly at 4° C. These samples and frozen brain slices were processed for immunohistochemical staining of neurons (mouse monoclonal NeuN antibody, Millipore, MAB377, mouse monoclonal β-III tubulin antibody, Thermo Fisher, 2G10, , mouse monoclonal glutamic acid decarboxylase antibody, Abcam, ab261113, all at 1:500 dilution, and rabbit polyclonal calmodulin kinase II antibody, Santa Cruz, sc13082 1:1000 dilution), astrocytes (rabbit polyclonal glial fibrillary acidic protein (GFAP) antibody. Abcam, ab7260 at 1:1000 dilution), and glia (Iba-1. Novachem, #019-19741 at 1:200 dilution). The cells were washed, permeabilized and blocked and incubated in primary antibody overnight at 4° C. on a 50 RPM rocker. Next day, the cells were washed with Tween buffer for 4×5 min (600 μL/well) before incubation in secondary antibodies (mouse Alexa 488, green (1:1000 dilution) and rabbit Alexa 568, red (1:1000 dilution)) for 1 hr at room temperature. The cells were washed and finally incubated in 4′,6-diamidino-2-phenylindole (DAPI) (1 μL/5 mL PBS) at room temperature for 5 min to stain nuclei. This step was followed by washing with PBS for 3×5 trins (600 μL/well) and proceeded with mounting in DAKO. Mounted slides were stored at 4° C. Images were obtained using a Nikon Eclipse confocal microscope, with excitation lasers at 405 nm (blue for DAPI), 488 urn (green for tubulin and NeuN) and 561 nm (red for GFAP) and a 60× or 100× oil-immersion objective.

### Results—4 Day and 7 Day Co-Culture Study

### Functional Testing by Electrophysiology

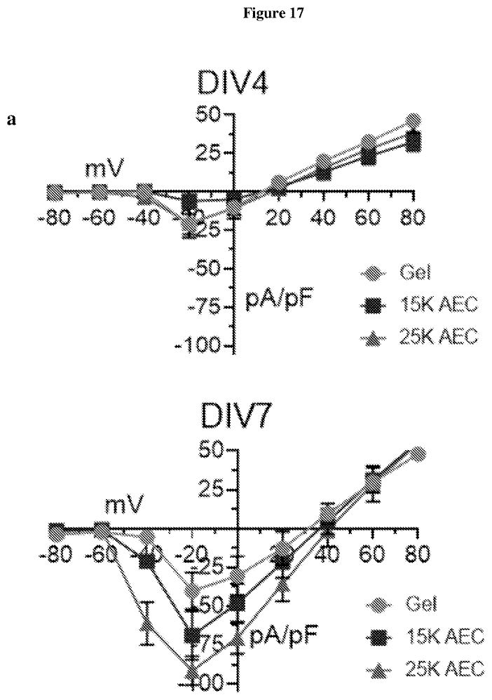

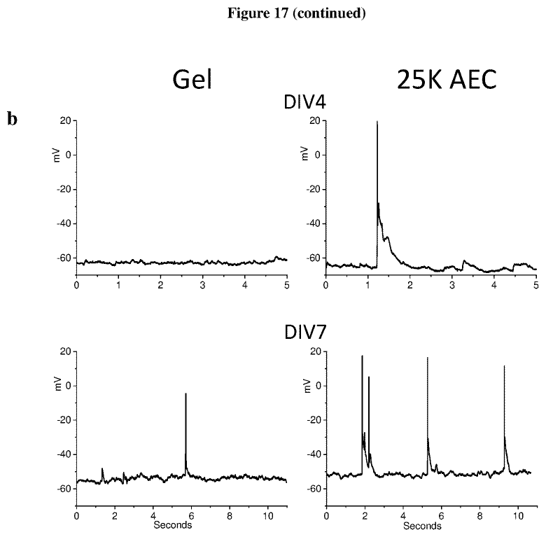

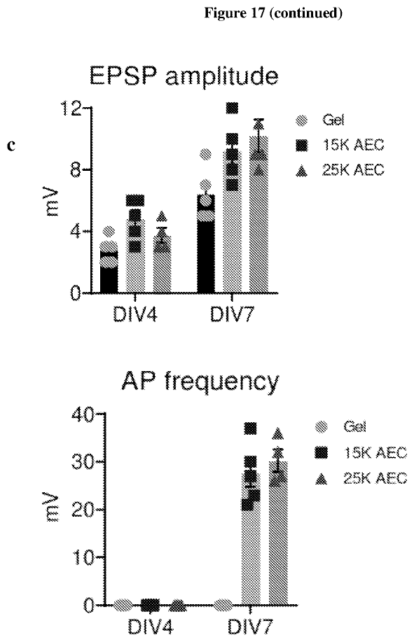

The results of the sodium current test are shown in FIG. 17a for the following treatments: gel (circles), hydrogel comprising 15,000 human amnion epithelial cells encapsulated within the hydrogel (15K AEC, squares) and hydrogel comprising 25,000 human amnion epithelial cells encapsulated within the hydrogel (25K AEC, triangles). After 4 days of in vitro co-culture (DIV4), primary hippocampal neurons showed small changes in sodium current (measured in pA/pF) in response to stimulus voltages (in mV). Through the period to 7 days of in vitro co-culture (DIV7), the primary hippocampal neurons exhibited larger increases in sodium current in response to a test pulse. This is illustrated by the 15 K AEC and 25 K AEC groups having larger sodium current values at specific stimulus voltages.

The results of the excitatory postsynaptic potential (EPSP) and action potential (AP) recordings are shown in FIGS. 17b and 17c for the following treatments: hydrogel (green circles), hydrogel comprising 15,000 human amnion epithelial cells encapsulated within the hydrogel (15K AEC, squares) and hydrogel comprising 25,000 human amnion epithelial cells encapsulated within the hydrogel (25K AEC, triangles). At DIV4, the primary hippocampal neurons co-cultured with hAEC hydrogel exhibited increased EPSP amplitudes. At DIV7, the primary hippocampal neurons co-cultured with hAEC hydrogel exhibited yet further increases in EPSP amplitudes as well as increased AP frequency.

### Anatomical Testing by Immunohistochemistry

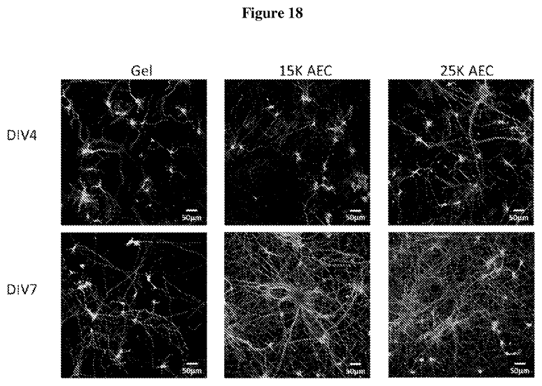

The primary hippocampal neuron co-cultures were subjected to immunofluorescent staining. GFAP was used to stain for astrocytes (in red, colour not shown), neuronal nuclear antigen (NeuN) used for staining neurons (in green, colour not shown) and DAPI for staining nuclei (in blue, colour not shown). FIG. 18 is a fluorescence microscopy image of the neuron co-culture wells at DIV4 and DIV7. There is a visible increase in astrocyte numbers starting at DIV4 and becoming more pronounced at DIV7, for both the 15 K AEC and 25 K AEC groups. For the 25 K AEC groups, there was a marked increase in astrocyte numbers at DIV7. The figure also shows that there was an increase in neuron branching and synaptic connectivity for both the 15 K AEC and 25 K AEC groups, as illustrated by the fluorescent green staining. This was evident at DIV4, and increased further at DIV7. In contrast, the hydrogel only group only showed slight neuronal branching.

Neurons communicate when one neuron releases a chemical (a neurotransmitter) to which another neuron responds. This communication takes place at the synapse. The first neuron in the conversation sends an action potential (AP) to the synapse and this AP causes the release of neurotransmitter. The transmitter rapidly traverses the synaptic cleft and creates an excitatory postsynaptic potential (EPSP) response in the receiving neuron. When the EPSP is large enough, it sets up an AP in the receiving neuron, completing the communication.

The above results illustrate that primary hippocampal neurons exposed to hydrogel-encapsulated hAECs exhibited increased sodium current and increased EPSP amplitudes, suggesting that hydrogel-encapsulated hAECs promoted development of the synapse between neurons, resulting in an increase in synaptic connectivity. Further, the immunofluorescence studies showed that hydrogel-encapsulated hAECs resulted in an increase in astrocyte number, and a concomitant increase in neuron branching and synaptic connectivity. This in turn has resulted in an increase in EPSP amplitudes and spontaneous AP frequency, indicating facilitation of communication between neurons. Astrocytes play a critically important role in supporting the development and function of the synapse. Taken together, the results show that hydrogel-encapsulated hAECs have direct effects on neuron function and anatomy resulting in improved synaptic connectivity, and indirectly support neuron function by promoting the number of astroglial support cells. This suggests that hydrogel-encapsulated hAECs may be useful in the treatment of diseases or conditions involving impairment to neuron function or neuronal death, such as stroke or neurodegenerative disorders.

### CITATION LIST

Bibliographic details of certain publications referred to in this specification are listed below alphabetically by author:
Alitalo, K., et al., Extracellular matrix components synthesized by human amniotic epithelial cells in culture. Cell, 1980. 19(4): p. 1053-1062.
Buckley, C. D., et al., RGD peptides induce apoptosis by direct caspase-3 activation. Nature, 1999. 397(6719): p. 534-9.
Holmes, T. C., et al., Extensive neurite outgrowth and active synapse formation on self-assembling peptide scaffolds. Proceedings of the National Academy of Sciences, 2000. 97(12): p. 6728-6733.
Kulkarni, K., et al., Orthogonal strategy for the synthesis of dual-functionalised [small beta]β-peptide based hydrogels. Chemical Communications, 2016. 52(34): p. 5844-5847 Liebmann, T., et al., Self-assembling Fmoc dipeptide hydrogel for in situ 3D cell culturing. BMC Biotechnology, 2007. 7(1): p. 88
Kulkarni, K., et al., β3-Tripeptides Coassemble into Fluorescent Hydrogels for Serial Monitoring in Vivo. ACS Biomaterials Science and Engineering, 2018. 4: p. 3843-8847. Manuelpillai, U., et al., Human Amniotic Epithelial Cell Transplantation Induces Markers of Alternative Macrophage Activation and Reduces Established Hepatic Fibrosis. PLoS One, 2012. 7(6).
Martin, A. D., et al., Controlling self-assembly of diphenylalanine peptides at high pH using heterocyclic capping groups. Scientific Reports, 2017. 7: p. 43947.
Mishra, A., et al., Influence of metal salts on the hydrogelation properties of ultrashort aliphatic peptides. RSC Advances, 2013. 3(25): p. 9985-9993.
Motamed, S., et al., A self-assembling beta-peptide hydrogel for neural tissue engineering. Soft Matter, 2016. 12(8): p. 2243-6;
Pomfret, R., et al., The Substitute Brain and the Potential of the Gel Model. Annals of Neurosciences, 2013. 20(3): p. 118-122.

It is to be understood that, if any prior art publication is referred to herein, such reference does not constitute an admission that the publication forms a part of the common general knowledge in the art, in Australia or any other country.

1. A β-peptide of formula (I): 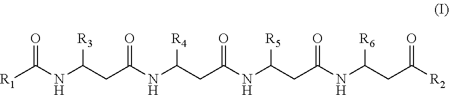
wherein:
R₁ is selected from —C₁₋₂₀alkyl, —C₂₋₂₀alkenyl, —C₂₋₂₀alkynyl, —OC₁₋₂₀alkyl, —OC₂₋₂₀alkenyl and —OC₂₋₂₀alkynyl;
R₂ is selected from —H, —OR₁₀ₐ, —SR₁₀ₐ, —N(R₁₁ₐ)₂, —NH(C═NH)NH₂, cycloalkyl, aryl, heterocyclyl, and heteroaryl, where each cycloalkyl, aryl, heterocyclyl, and heteroaryl is unsubstituted or is substituted with one or more groups selected from —OH, —NH₂, —NHC₁₋₃alkyl, —OC₁₋₃alkyl, —SH, —SC₁₋₃alkyl, —CO₂H, —CO₂C₁₋₃alkyl, —CONH₂ and —CONHC₁₋₃alkyl, or R₂ has the following structure (A): 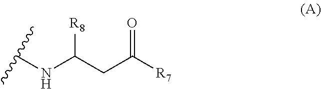
R₃, R₄, R₅, R₆ and R₈ are each independently selected from —H, —R₉, —C₁₋₆alkylR₉, —C₂₋₆alkenylR₉ and —C₂₋₆alkynylR₉;
or R₂ and R₆ together form a heterocyclic ring;
R₇ is selected from —H, —OR₁₀ₐ, —SR₁₀ₐ, —N(R₁₁ₐ)₂, —NH(C═NH)NH₂, cycloalkyl, aryl, heterocyclyl, and heteroaryl, where each cycloalkyl, aryl, heterocyclyl, and heteroaryl is unsubstituted or is substituted with one or more groups selected from —OH, —NH₂, —NHC₁₋₃alkyl, —OC₁₋₃alkyl, —SH, —SC₁₋₃alkyl, —CO₂H, —CO₂C₁₋₃alkyl, —CONH₂ and —CONHC₁₋₃alkyl;
or R₇ and R₈ together form a heterocyclic ring;
R₉ is selected from —H, —OR₁₀, —SR₁₀, —N(R₁₁)₂, —C(O)R₁₂, —NH(C═NH)NH₂, cycloalkyl, aryl, heterocyclyl, and heteroaryl, where each cycloalkyl, aryl, heterocyclyl, and heteroaryl is unsubstituted or is substituted with one or more groups selected from —OH, —NH₂, —NHC₁₋₃alkyl, —OC₁₋₃alkyl, —SH, —SC₁₋₃alkyl, —CO₂H, —CO₂C₁₋₃alkyl, —CONH₂ and —CONHC₁₋₃alkyl;
R₁₀ is selected from —H, —C₁₋₆alkyl, —C₂₋₆alkenyl, —C₂₋₆alkynyl, —C(O)C₁₋₂₀alkyl, —C(O)C₁₋₂₀alkenyl, —C(O)C₁₋₂₀alkynyl, —C(O)C₁₋₆alkylR₁₄, an α-peptide having 1 to 10 α-amino acid residues and an imaging agent;
R₁₀ₐ is selected from —H, —C₁₋₆alkyl, —C₂₋₆alkenyl, —C₂₋₆alkynyl, —C(O)C₁₋₆alkyl, —C(O)C₂₋₆alkenyl, and —C(O)C₂₋₆alkynyl;
each R₁₁ is independently selected from —H, —C₁₋₆alkyl, —C₁₋₆alkylN(R₁₃)₂, μ-C₁₋₆alkylNR₁₃R₁₄, —C₂₋₆alkenyl, —C₂₋₆alkynyl, —C(O)C₁₋₂₀alkyl, —C(O)C₁₋₂₀alkenyl, —C(O)C₁₋₂₀alkynyl, —C(O)C₁₋₆alkylR₁₄, an α-peptide having 1 to 10 α-amino acid residues and an imaging agent;
each R₁₁ₐ is independently selected from —H, —C₁₋₆alkyl, —C₂₋₆alkenyl, —C₂₋₆alkynyl, —C(O)C₁₋₆alkyl, —C(O)C₂₋₆alkenyl, —C(O)C₂₋₆alkynyl;
R₁₂ is selected from —OH, —OR₁₀ and —N(R₁₁)₂;
each R₁₃ is independently selected from —H and —C₁₋₆alkyl;
R₁₄ is an α-peptide having 1 to 10 α-amino acid residues or an imaging agent;
or a pharmaceutically acceptable salt thereof;
and wherein one of R₁, R₂, R₃, R₄, R₈, R₆ and R₈ comprise a C₈₋₂₀alkyl group, a C₈₋₂₀alkenyl group or a C₈₋₂₀alkynyl group.

2-32. (canceled)

33. The β-peptide according to claim 1 wherein R₁ is C₁₋₂₀alkyl.

34. The β-peptide according to claim 33 wherein R₁ is methyl.

35. The β-peptide according to claim 1 wherein R₂ is —OH or wherein R₂ together with R₆ forms a heterocycle.

36. The β-peptide according to claim 35, wherein R₂ together with R₆ together form a lactone.

37. The β-peptide according to claim 36, wherein the β-peptide has the formula:

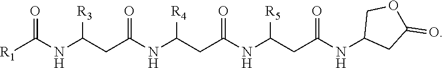

38. The β-peptide according to claim 1 wherein one or more of R₃, R₄, R₅, R₆ and R₈ corresponds to an amino acid side chain, where each amino acid side chain may be the same or different.

39. The β-peptide according to claim 1 wherein one or more of R₃, R₄, R₅ and R₆ corresponds to a hydrophilic amino acid side chain, where each hydrophilic amino acid side chain may be the same or different.

40. The β-peptide according to claim 1 wherein one of R₃, R₄, R₇, R₆ or R₈ comprises a C₈₋₂₀alkyl group.

41. The β-peptide according to claim 1 wherein one or more of R₃, R₄, R₅, R₆ comprises an α-peptide having 1 to 10 α-amino acid residues.

42. The β-peptide according to claim 1 wherein one or more of R₃, R₄, R₅, R₆ comprises an imaging agent.

43. The β-peptide according to claim 1, wherein the β-peptide is selected from the following:

| No. | Structure |
|---|---|
| 1 | 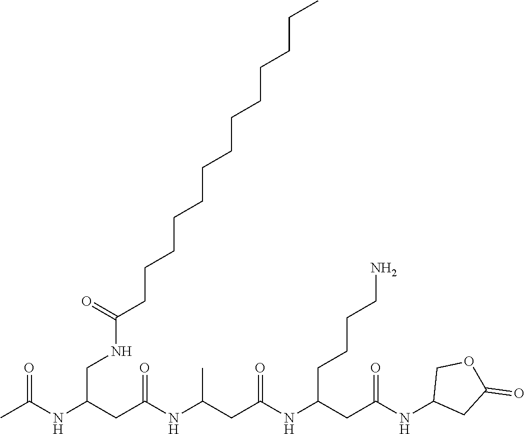 [SMILES: CCCCCCCCCCCCCC(=O)NCC(CC(=O)NC(C)CC(=O)NC(CCCCN)CC(=O)NC1COC(=O)C1)NC(C)=O] |
| 2 | 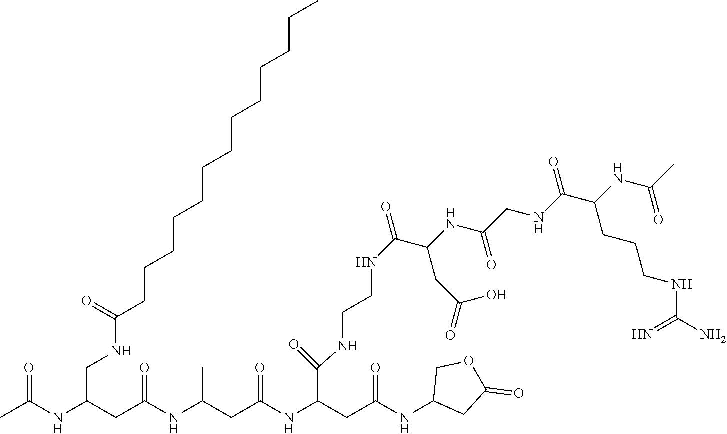 [SMILES: CCCCCCCCCCCCCC(=O)NCC(CC(=O)NC(C)CC(=O)NC(CC(=O)NC1COC(=O)C1)C(=O)NCCNC(=O)C(CC(=O)O)NC(=O)CNC(=O)C(CCCNC(=N)N)NC(C)=O)NC(C)=O] |
| 3 | 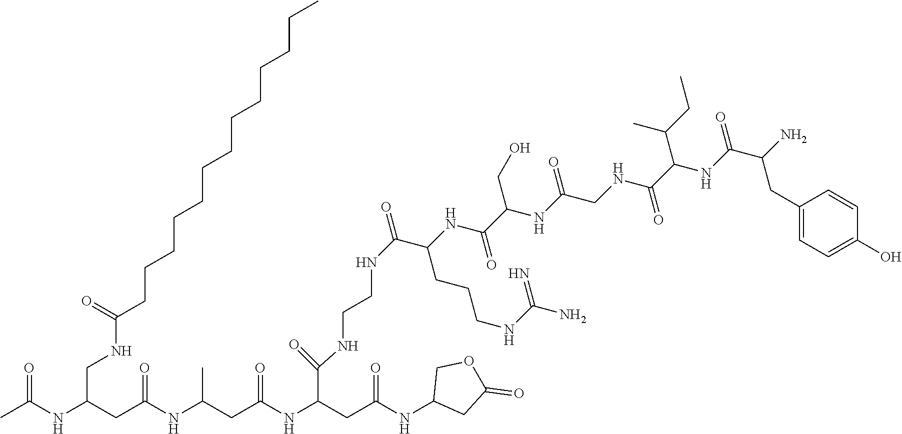 [SMILES: CCCCCCCCCCCCCC(=O)NCC(CC(=O)NC(C)CC(=O)NC(CC(=O)NC1COC(=O)C1)C(=O)NCCNC(=O)C(CCCNC(=N)N)NC(=O)C(CO)NC(=O)CNC(=O)C(NC(=O)C(N)Cc1ccc(O)cc1)C(C)CC)NC(C)=O] |
| 4 | 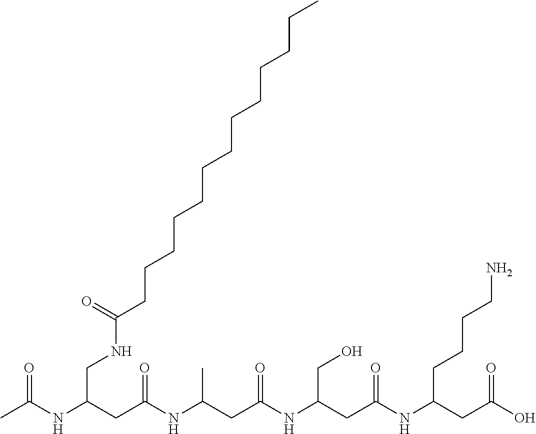 [SMILES: CCCCCCCCCCCCCC(=O)NCC(CC(=O)NC(C)CC(=O)NC(CO)CC(=O)NC(CCCCN)CC(=O)O)NC(C)=O] |
| 5 | 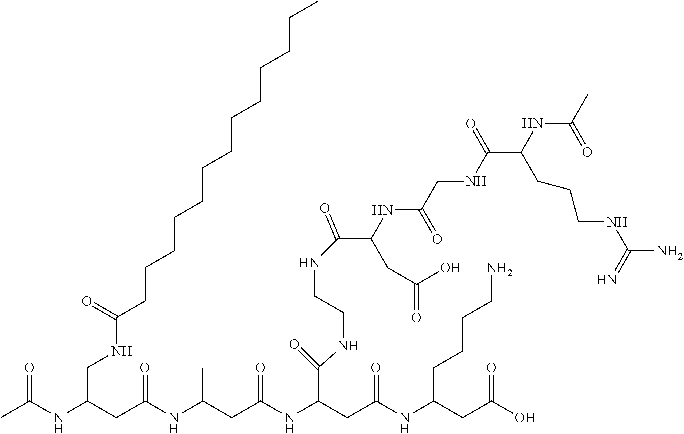 [SMILES: CCCCCCCCCCCCCC(=O)NCC(CC(=O)NC(C)CC(=O)NC(CC(=O)NC(CCCCN)CC(=O)O)C(=O)NCCNC(=O)C(CC(=O)O)NC(=O)CNC(=O)C(CCCNC(=N)N)NC(C)=O)NC(C)=O] |
| 6 | 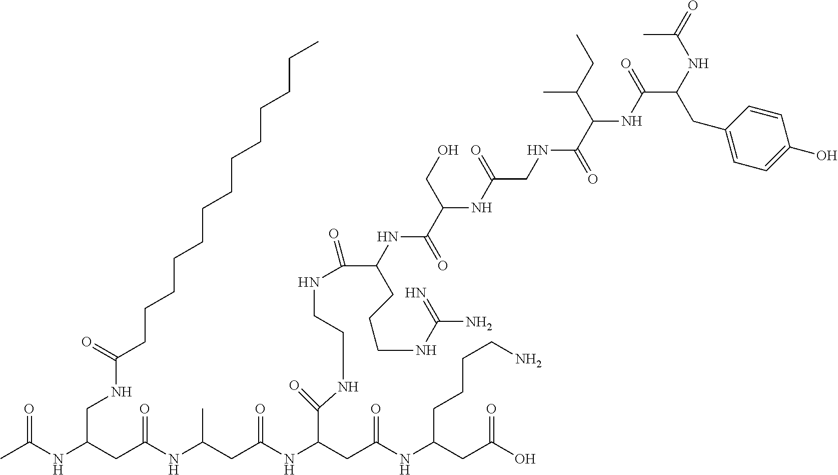 [SMILES: CCCCCCCCCCCCCC(=O)NCC(CC(=O)NC(C)CC(=O)NC(CC(=O)NC(CCCCN)CC(=O)O)C(=O)NCCNC(=O)C(CCCNC(=N)N)NC(=O)C(CO)NC(=O)CNC(=O)C(NC(=O)C(Cc1ccc(O)cc1)NC(C)=O)C(C)CC)NC(C)=O] |
| 7 | 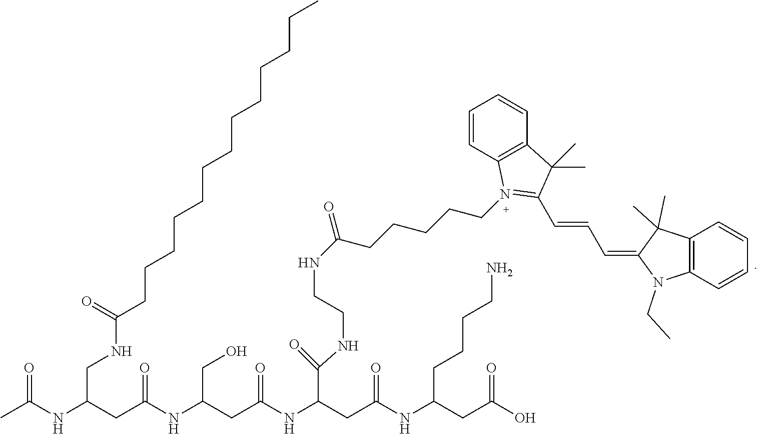 |

44. A hydrogel comprising one or more β-peptides of formula (I) according to claim 1.

45. The hydrogel according to claim 44 wherein the hydrogel comprises one β-peptide of formula (I) or two β-peptides of formula (I) which are a first β-peptide and a second β-peptide.

46. The hydrogel according to claim 45 wherein the hydrogel comprises a first β-peptide and a second β-peptide in a ratio of 3:1, 2:1, 1:1, 1:2, or 1:3 by weight.

47. The hydrogel according to claim 44 wherein the hydrogel further comprises a therapeutic cargo encapsulated within the hydrogel.

48. The hydrogel according to claim 47 wherein the therapeutic cargo is selected from a small molecule drug, a macromolecular drug and a cell.

49. The hydrogel according to claim 48 wherein the cell is a stem cell.

50. The hydrogel according to claim 44 wherein the hydrogel is injectable.

51. A method for treating stroke comprising administering to a patient in need thereof a hydrogel comprising:
one or more β-peptides of formula (I) according to claim 1; and
a stem cell encapsulated within the hydrogel.
